(19) United States
(12) Patent Application Publication
(10) Pub. No.: US 2026/0347787 A1
(43) Pub. Date: Nov. 5, 2026

(54) COMPOSITIONS AND METHODS FOR OBTAINING SUSPENSION HUMAN INTESTINAL ORGANOIDS HAVING A SEROSAL MESOTHELIAL LAYER

(71) Applicant: The Regents of the University of Michigan, Ann Arbor, MI (US)

(72) Inventors: Jason Spence, Ann Arbor, MI (US); Meghan Capeling, Ann Arbor, MI (US)

(21) Appl. No.: 19/111,821

(22) PCT Filed: Sep. 14, 2023
(86) PCT No.: PCT/US2023/032761

§ 371 Date: Mar. 14, 2025

Related U.S. Application Data
(60) Provisional application No. 63/406,498, filed on Sep. 14, 2022.

(51) Int. Cl. C12N 5/071; A61K 35/38

(52) U.S. Cl. CPC C12N 5/0679; A61K 35/38; C12N 2501/11; C12N 2501/155; C12N 2501/415; C12N 2513/00

(57) ABSTRACT

The invention disclosed herein generally relates to methods and systems for growing, expanding and/or obtaining human intestinal organoids (HIOs) having a serosal mesothelial layer. In particular, the invention disclosed herein relates to methods and systems for culturing hindgut spheroid tissue in suspension to obtain HIOs having an organized serosal mesothelial layer that is similar to primary human intestinal serosal mesothelium.

### STATEMENT OF RELATED APPLICATIONS

This application claims priority to U.S. Provisional Patent Application No. 63/406,498, filed Sep. 14, 2023, the entire contents of which are incorporated herein by reference for all purposes.

### FIELD OF THE INVENTION

The invention disclosed herein generally relates to methods and systems for growing, expanding and/or obtaining human intestinal organoids (HIOs) having a serosal mesothelial layer. In particular, the invention disclosed herein relates to methods and systems for culturing hindgut spheroid tissue in suspension to obtain HIOs having an organized serosal mesothelial layer that is similar to primary human intestinal serosal mesothelium.

### INTRODUCTION

Human intestinal organoids (HIOs) are 3D tissues derived from human pluripotent stem cells (hPSCs) that mimic the structure and function of the human intestine (McCracken et al., 2011; Spence et al., 2011). However, HIOs lack some key cell types found in the native intestine including vasculature and neurons (Finkbeiner et al., 2015; Holloway et al., 2020; Schlieve et al., 2017; Watson et al., 2014; Workman et al., 2016). HIOs offer advantages over 2D cell-based model systems that do not recapitulate the 3D architecture of human organs, or animal models that do not always mimic human physiology (Dedhia et al., 2016). While HIOs are a promising tool to study intestinal development and disease, they have been hindered by reliance on basement membrane extracellular matrix (ECM) products (i.e. Matrigel). These ECMs support growth but introduce biological variability and are not amenable to clinical applications due to high cost and xenogeneic origin (Czerwinski and Spence, 2017).

Improved methods for generating HIOs that are not reliant on basement membrane ECM products and more closely recapitulate the complex cell types found within the human intestine are needed.

The present invention addresses this need.

### SUMMARY OF THE INVENTION

It has been recently demonstrated that HIOs, which possess both epithelium and mesenchyme, create their own basement membrane and can thus be cultured in biologically inert alginate hydrogels (Capeling et al., 2019). The observation that HIOs can grow in the absence of a biochemically supportive ECM led to a hypothesis that HIOs may not require extrinsic support.

Experiments conducted during the course of developing embodiments for the present invention demonstrated that a 3D substrate is dispensable for HIO culture and that HIOs can be cultured in suspension. This technique increases the simplicity of HIO culture, streamlines maintenance while enhancing the ability to scale-up, and reduces experimental cost and variability.

The serosal mesothelium is the outermost layer of the intestine comprised of a single layer of squamous mesothelial cells (Bloom and Fawcett, 1975). It provides a protective boundary for the intestine and creates a lubricating, frictionless surface (Mutsaers, 2004) that is involved in homeostasis and disease (Bridda et al., 2007; Dobbie, 1992; Federici et al., 2003; Pumberger et al., 2002; Sulaiman et al., 2001; Waldhausen and Sawin, 1997; Winters and Bader, 2013). The serosa also plays a critical role in development via contributions to mesenchyme, especially vascular smooth muscle, in multiple organs (Carmona et al., 2013; Dettman et al., 1998; Que et al., 2008; Wilm et al., 2005). Despite the need to better understand this tissue, a serosal mesothelium has never been described in complex 3D models of the human intestine.

Experiments conducted during the course of developing embodiments for the present invention further demonstrated that while HIOs cultured in suspension possessed expected epithelial cell types, they additionally exhibited enhanced mesenchymal organization including a serosal mesothelial-like layer.

Accordingly, the invention disclosed herein generally relates to methods and systems for growing, expanding and/or obtaining human intestinal organoids (HIOs) having a serosal mesothelial layer. In particular, the invention disclosed herein relates to methods and systems for culturing hindgut spheroid tissue in suspension to obtain HIOs having an organized serosal mesothelial layer that is similar to primary human intestinal serosal mesothelium.

In certain embodiments, the present invention provides methods of inducing formation of suspension HIOs having a serosal mesothelial layer. These methods comprise obtaining HIOs using established protocols, and culturing the obtained HIOs in suspension. In some embodiments, the culturing of the obtained HIOs in suspension results in suspension HIOs and in vitro differentiation of an organized serosal mesothelial layer (e.g., suspension HIOs having a serosal mesothelial layer).

Such embodiments are not limited to a particular manner of culturing the obtained HIOs in suspension. In some embodiments, the culturing of the obtained HIOs in suspension comprises seeding the obtained HIOs with a growth medium. Such embodiments are not limited to a particular type of growth medium. In some embodiments, the growth medium comprises one or more of the following: Advanced Dulbecco's Modified Eagle Medium/Nutrient Mixture F-12 (DMEM/F12) supplemented with 4-(2-hydroxyethyl)-1-piperazineethanesulfonic acid (HEPES), GlutaMAX, Penicillin/Streptomycin, epidermal growth factor (EGF), a BMP inhibiting agent (e.g., Noggin), and R-Spondin. In some embodiments, the growth medium comprises Advanced DMEM/F12 supplemented with HEPES, GlutaMAX, Penicillin/Streptomycin, EGF, Noggin, and R-Spondin.

In some embodiments, seeding the obtained HIOs with the growth medium comprises plating the obtained HIOs in low attachment plates with the growth medium such that the HIOs remain in suspension instead of adhering to the culture dish.

In some embodiments, seeding the obtained HIOs with the growth medium comprises plating the obtained HIOs in low attachment plates with the growth medium having EGF, Noggin, and R-spondin for approximately 72 hours (e.g., 65 hours, 66, 67, 68, 69, 70, 71, 72, 73, 74, 75, 76, 77, 78, 79 hours) followed by culturing with EGF for remaining culture time.

Such methods are not limited to a particular BMP inhibiting agent within the growth medium. In some embodiments, the BMP inhibiting agent is a small molecule or antagonist that inhibits the BMP signaling pathway. In some embodiments, the small molecule or antagonist that inhibits the BMP signaling pathway is Noggin.

In some embodiments, the suspension HIOs having a serosal mesothelial layer comprise an inner ECAD+ epithelium surrounded by VIM+ mesenchyme. In some embodiments, the suspension HIOs having a serosal mesothelial layer comprise expression of one or more of DPP4+ enterocytes, MUC2+ goblet cells, and CHGA+ enteroendocrine cells.

In some embodiments, the serosal mesothelial layer of the suspension HIOs express one or more of WT1, UPK3B, MSLN, and KRT19. In some embodiments, the serosal mesothelial layer of the suspension HIOs express one or more of CAV1, CAV2, and EZR.

In some embodiments, the suspension HIOs having a serosal mesothelial layer comprise an epithelium and/or mesenchyme. In some embodiments, the epithelium expresses one or both of CDX2 and PDX1. In some embodiments, the suspension HIOs having a serosal mesothelial layer comprise epithelium and mesenchyme, wherein the epithelium and mesenchyme express KI67. In some embodiments, the epithelium expresses SOX9.

In some embodiments, the suspension HIOs having a serosal mesothelial layer comprise an apical surface. In some embodiments, the apical surface expresses ZO-1.

In certain embodiments, the present invention provides suspension HIOs having an organized serosal mesothelial layer.

In certain embodiments, the present invention provides methods of treating a mammalian subject having a damaged intestinal tissue with reduced function by engrafting at the site of injury suspension HIOs having an organized serosal mesothelial layer.

In some embodiments, the damaged intestinal tissue includes serosal adhesions occurring following abdominal surgery.

In some embodiments, the methods comprise the steps of administering the suspension HIOs having an organized serosal mesothelial layer to the subject, wherein the suspension HIOs having an organized serosal mesothelial layer engraft at the site of injury and repopulate at least a portion of the site with the suspension HIOs having an organized serosal mesothelial layer, wherein the repopulated suspension HIOs having an organized serosal mesothelial layer supplement the function, thereby treating the subject.

In certain embodiments, the present invention provides methods of treating a mammalian subject having a damaged intestinal tissue with reduced function, comprising:
a) culturing HIOs in suspension, wherein the culturing in suspension results in suspension HIOs and in vitro differentiation of an organized serosal mesothelial layer;
b obtaining suspension HIOs having an organized serosal mesothelial layer;
c) engrafting the obtained suspension HIOs having an organized serosal mesothelial layer at the site of injury, wherein engrafted suspension HIOs having an organized serosal mesothelial layer engraft at the site of injury and repopulate at least a portion of the site with the engrafted cells, wherein the repopulated cells supplement the function, thereby treating the subject.

In some embodiments, the obtained HIOs used in the culturing in suspension embodiments are derived from intestinal hindgut spheroids. In some embodiments, the obtained HIOs used in the culturing in suspension embodiments are pluripotent stem cell-derived human intestinal organoids

In some embodiments, the culturing and obtaining steps are conducted in vitro.

In certain embodiments, the present invention provides compositions comprising suspension HIOs having an organized serosal mesothelial layer produced in vitro from the described methods.

In certain embodiments, the present invention provides kits comprising suspension HIOs having an organized serosal mesothelial layer produced in vitro from the described methods.

### BRIEF DESCRIPTION OF DRAWINGS

FIG. 1. Suspension culture supports HIO growth and serosa formation. (a): Schematic depicting HIO suspension culture. Right: Hematoxylin and eosin (H&E) stain of a 28-day suspension HIO. The epithelium is outlined in green. (b): Representative images of HIOs cultured in suspension for 28 days. Markers shown are ECAD, CDX2, PDX1, VIM, and ZO-1. Scale bar=50 μm. (c): Representative images of epithelial cell marker staining in HIOs cultured in suspension for 28 days. Markers shown are SOX9, KI67, DPP4, MUC2, and CHGA. Scale bar=50 μm. (d): H&E staining of the outer mesenchymal layer in human fetal intestine compared to HIOs cultured in Matrigel, 1% alginate, and suspension for 28 days. Scale bar=25 μm. (e): Representative images of mesothelial markers in human fetal intestine compared to HIOs cultured in Matrigel, 1% alginate, and suspension for 28 days. Markers shown are WT1, pan-Cytokeratin (pCK), LAM, and ECAD. Scale bar=100 μm.

FIG. 2. Suspension HIOs resemble alginate and Matrigel-grown HIOs in vitro and in vivo. Related to FIG. 1. (a): Quantification of HIO yield after 28 days. HIO yield was calculated as the percentage of spheroids which gave rise to HIOs. Data shown are average yields from 4 independent experiments (in suspension, biological replicates) with n>200 spheroids per condition, compared to previously published data for alginate and Matrigel (Capeling et al., 2019). Each point depicts mean yield from one experiment, while bars depict mean and standard error. Significance was calculated with a one-way ANOVA and multiple comparisons test. (b): qRT-PCR analysis of CDX2, PDX1, MUC2, CHGA, KI67, and LGR5 expression in HIOs derived from 3 independent hPSC lines cultured in 1% alginate, Matrigel, and suspension for 28 days in vitro (biological replicates. Expression levels are normalized to ECAD expression to account for varying amounts of epithelium in HIOs. Each point is representative of 6-10 HIOs pooled from the same batch. Data represent the mean±standard error of the mean. Significance was calculated with a one-way ANOVA and multiple comparisons test. (c): qRT-PCR analysis of CDX2, MUC2, CHGA, and LGR5 expression in HIOs derived from 2 independent hPSC lines cultured in suspension for 28 days in vitro (biological replicates) compared to 11-week human fetal intestine (technical replicates). Expression levels are normalized to ECAD expression to account for varying amounts of epithelium. Data represent the mean±standard error of the mean. Significance was calculated with a one-way ANOVA and multiple comparisons test. (d): Hematoxylin and eosin staining in suspension HIOs cultured for 28 days in vitro and then transplanted beneath the kidney capsule of immunocompromised mice for 8 weeks in vivo. Scale bar=100 μm. (e): Representative images of epithelial cell marker staining in suspension tHIOs. Markers shown are ECAD, CDX2, PDX1, VIM, ZO1, SOX9, KI67, DPP4, MUC2, CHGA, LYZ (Paneth cell marker), DEFA5 (Paneth cell marker), and OLFM4 (intestinal stem cell marker). Scale bar=50 μm.

FIG. 3. Characterization and scRNA-seq analysis of human fetal small intestine and suspension HIOs. Related to FIGS. 1 and 4. (a): Left: Transmission electron micrograph depicting microvilli lining on the surface of a 28-day suspension HIO. Right: Immunofluorescent image of VIL1 marker staining in the ECAD+ mesothelium of a 28-day suspension HIO. (b): Results from an ELISA for t-PA concentration in conditioned media collected from 28-day HIOs cultured in Matrigel and suspension. (c): Left: UMAP plot of 51,790 cells extracted from 8 human fetal small intestinal samples (duodenum, jejunum, and ileum) ranging from day 47 to day 132 profiled with scRNA-seq predicted 15 cell clusters. Data were visualized using Uniform Manifold Approximation and Projection (UMAP) dimensional reduction. The batch correction algorithm BBKNN was utilized to account for batch differences between samples. Right: UMAP plot of human fetal intestinal samples indicating sample age of individual cells. (d): Dotplot of genes in human fetal intestinal samples associated with major cell classes: epithelial, mesenchymal, neuronal, endothelial, immune, and mesothelial. Dot size denotes the proportion of cells in a given cluster expressing each marker. Dot color indicates expression level within the cluster based on normalized z-score. Clusters are colored by class assignment: epithelium=yellow, mesenchyme=red/pink, neuronal=light blue, endothelial=green, immune=purple, serosa/mesothelial=dark blue. (e): Feature plots for mesothelial-specific markers WT1, MSLN, and UPK3B, highlighting the mesothelial population within a subset of cluster 8. (f): Feature plot for mesothelial-specific markers WT1 and dot plot for MSLN, WT1, UPK3B, and KRT19 in an analysis of 4,619 cells from 28-day suspension HIOs (FIG. 4B), highlighting mesothelial gene expression within a subset of cluster 2. (g): Representative images of non-canonical mesothelial marker staining in human fetal intestine and suspension HIOs. Markers shown are ECAD, EZR, CAV1, and CAV2. Scale bar=50 μm. (h): Feature plots and dotplots for mesothelial markers GATA4, TCF21, and TBX18 depicting expression in extracted clusters of human fetal intestinal serosa as well as HIO-serosa. We observed expression of the mesothelial marker TCF21 in both human and HIO-serosa but did not observe high expression of the mesothelial markers GATA4 or TBX18 in either population, which may highlight organ and/or species-specific differences in mesothelial gene expression as these markers have been more commonly described in mesothelial cells of the mouse lung and heart.

FIG. 4. HIO-serosa is molecularly and functionally similar to human serosa. (a): Left: UMAP plot of 761 cells computationally extracted from a sub-cluster within cluster 8 of the analysis on human fetal small intestine (FIG. 3). Right: Dotplot of mesothelial genes highlighting the mesothelial population within cluster 2 (human fetal serosa). For all dotplots in FIG. 4, dot size denotes the proportion of cells in a cluster expressing each marker, and dot color indicates expression level within the cluster based on normalized z-score. (b): Left: UMAP plot of 4,619 cells from 28-day suspension HIOs. Right: Dotplot of genes in suspension HIOs associated with major cell classes. (c): Left: UMAP plot of 989 cells computationally extracted from cluster 2 of the analysis on suspension HIOs (FIG. 4B). Right: Dotplot of mesothelial genes highlighting the mesothelial population within cluster 0 (HIO-serosa). (d): Venn diagram depicting genes that are significantly enriched (log₂ fold >1.5 in expression of a gene in the serosa cluster relative to all other clusters) in human fetal serosa (cluster 2, FIG. 4A) and suspension HIO-serosa (cluster 0, FIG. 4C). (e): Representative images of mesothelial markers used for FACS sorting in 28-day suspension HIOs (ECAD, EPCAM, and PDPN). Scale bar=50 μm. (f): qRT-PCR analysis of WT1 and UPK3B expression in FACS-isolated serosa, mesenchyme, and epithelium from suspension HIOs. (g): Representative images of FACS-sorted HIO-serosa stained for WT1 and αSMA after 24 hours or 7 days cultured in vitro. Scale bar=50 μm. (h): Quantification of the percentage of DAPI+ FACS-sorted HIO-serosa cells that express αSMA. Each point represents 1 independent experiment with cells isolated from suspension HIOs from 3 different hPSC lines. (i): qRT-PCR analysis of ACTA2 and TAGLN expression in FACS-sorted HIO-serosa cells immediately after sorting or after 7 days in culture. For all graphs in FIG. 4, data represent the mean±standard error of the mean. Significance was calculated with a one-way ANOVA and multiple comparisons test. For all qRT-PCR data, expression levels are normalized to GAPDH. Each point is representative of sorting results from one pooled batch of HIOs, across n=3 independent experiments (biological replicates).

FIG. 5. Validation of HIO-serosa FACS isolation and Extraction of serosa cluster from HIO time course. Related to FIGS. 4 and 8. (a): Representative images of mesothelial markers used for FACS sorting in human fetal intestine. Markers shown are ECAD, EPCAM, and PDPN. Scale bar=50 μm. (b): FACS plots demonstrating sorting strategy to isolate serosa, mesenchyme, and epithelium from suspension HIOs. Cells were first gated on PDPN. PDPN+/− cells were then sorted based on ECAD and EPCAM, and PDPN+/ECAD+/EPCAM− cells were collected as serosa, while PDPN−/ECAD−/EPCAM− cells were collected as mesenchyme, and PDPN−/ECAD+/EPCAM+ cells were collected as epithelium. (c): qRT-PCR analysis of ECAD and VIM expression in FACS-isolated serosa, mesenchymal, and epithelial populations from 3 independent batches of suspension HIOs. Expression levels are normalized to GAPDH. Each point is representative of sorting results from one pooled batch of HIOs. Data represent the mean±standard error of the mean. Significance was calculated with a one-way ANOVA and multiple comparisons test. (d): Bright field images of FACS-sorted serosa, mesenchymal, and epithelial populations from suspension HIOs cultured in basal media. Scale bar=100 μm. (e): Representative images of FACS-sorted mesenchyme and epithelium from suspension HIOs stained for WT1 and pCK after 24 hours in culture in basal media. Scale bar=50 μm. (f): Left: UMAP plot of 52,689 cells HIOs cultured in alginate and Matrigel collected at day 3, 7, 14, and 28 profiled with scRNAseq predicted 19 cell clusters. Right: Feature plots for mesothelial markers WT1 and UPK3B in the alginate/Matrigel HIO time course dataset depicting a serosal mesothelial population at the boundary between cluster 3 and cluster 4. (g): Left: UMAP plot of 8,179 cells extracted from clusters 3 and 4 of the analysis in FIG. 5A. Right: Feature plots for mesothelial markers WT1 and UPK3B in the analysis on extracted clusters 3 and 4 depicting a serosal mesothelial population in sub-cluster 4.

FIG. 6. Comparing HIO-serosa between Matrigel, alginate, and suspension culture. (a): UMAP plot of 13,055 cells from 28-day alginate, Matrigel, and suspension HIOs. The batch correction algorithm BBKNN was utilized. Feature plots for mesothelial markers WT1, MSLN, and UPK3B, highlighting the mesothelial population within cluster 3. (b): Left: UMAP plot of 1,903 cells computationally extracted from cluster 3 of the analysis on alginate, Matrigel, and suspension HIOs (FIG. 6A). Middle: Feature plots for mesothelial markers WT1, MSLN, and UPK3B, highlighting the mesothelial population within cluster 1. Right: Dotplot of mesothelial genes. (c): Left: UMAP plot of computationally extracted HIO-serosa (cluster 3, sub-cluster 1) separated by culture condition. Right: Quantification of the percentage of cells within cluster 3, sub-Cluster 1 that are derived from each condition. (d): Distribution of the cells from alginate, Matrigel, and suspension HIOs in cluster 3 that were extracted into sub-clusters 0, 1, or 2. (e): Quantification of the percentage of HIOs in 4 independent batches that form a full or partial WT1+pCK+ outer serosa. Each dot represents data from one batch, each color dot depicts an independent batch of HIOs (biological replicates), and bars depict mean and standard error. Significance was calculated with a one-way ANOVA and multiple comparisons test. (f): qRT-PCR analysis of WT1 and UPK3B expression in HIOs derived from 2 independent hPSC lines cultured in suspension for 28 days in vitro. Expression levels are normalized to GAPDH. Each point is representative of 6-10 HIOs pooled from the same batch (biological replicates). Data represent the mean±standard error of the mean. Significance was calculated with a one-way ANOVA and multiple comparisons test.

FIG. 7. Analysis of cell types within HIOs by culture condition. Related to FIG. 6. A total of 13,055 cells from 28-day HIOs cultured in Matrigel, alginate, and suspension were included in the computational analysis and the data were visualized using UMAP dimensional reduction (FIG. 6A, FIG. 7A). The batch correction algorithm BBKNN was utilized to account for batch differences between suspension, alginate, and Matrigel-grown HIOs. (a): Dotplot of genes in human fetal intestinal samples associated with major cell classes: epithelial, mesenchymal, and a small neuronal population in cluster 7. Dot size denotes the proportion of cells in a given cluster expressing each marker. Dot color indicates expression level within the cluster based on normalized z-score. Clusters are colored by class assignment: epithelium=yellow, mesenchyme=red/pink, neuronal=light blue. (b): Analysis of the contribution to each cluster based on HIO culture condition. Data shown are the number of cells in each cluster that came from HIOs cultured in suspension vs. alginate vs. Matrigel as well as the percentage of cells in each culture condition that were sorted into individual clusters. (c): Visual representation of the percentage of cells in each HIO culture condition that were sorted into clusters 0-7 using data from the table in FIG. 7B. (d): Comparison of the proportion of cells from alginate and suspension HIOs in each cluster, normalized to Matrigel HIOs. Notably, the proportion of cells in epithelial cluster 0 was comparable between HIO culture conditions while there were differences in the contribution to mesenchymal clusters.

FIG. 8. Signaling pathways implicated in serosa formation. (a): UMAP plot of 8,179 cells extracted from clusters 3 and 4 (alginate and Matrigel HIO time-course, FIG. 5). Right: Quantification of the percentage of cells within the serosal sub-cluster 4 that originate from HIOs collected at day 3, 7, 14, and 28. (b): Representative image of suspension HIOs collected at day 7 and 11 stained for WT1 and pCK. Scale bar=100 μm. (c): Qualitative scoring system to rank HIOs based on WT1 and pCK expression. 0=no serosa, 1=partial serosa, 2=complete serosa, 3=full or partial serosa plus ectopic WT1 expression in mesenchyme. Scale bar=100 μm. (d): Graph depicting the percentage of HIOs in control basal media that received each score (0-3) across 4 independent experiments (biological replicates). (e): Comparison of the percentage of HIOs that formed a complete serosa (score 2) in basal media to HIOs treated with inhibitors SU5402 (FGF), Noggin (BMP), DAPT (Notch), IWR1 (WNT), and Cyclopamine (HH). Significance was calculated in relation to the control basal media condition. (f): Quantification of the percentage of HIOs from 4 independent experiments that scored 0-3 in basal media or media supplemented with SAG or Cyclopamine. Right: Comparison of the percentage of HIOs that received score 2 in basal media compared to SAG and Cyclopamine treatment. (g): Quantification of the percentage of HIOs from 3 independent experiments that scored 0-3 in basal media or media supplemented with IWR1 or CHIR-99021. Right: Comparison of the percentage of HIOs that received score 2 in basal media compared to IWR1 and CHIR-99021 treatment. For all graphs in FIG. 8, data represent the mean±standard error of the mean. Significance was calculated with a one-way ANOVA and multiple comparisons test.

FIG. 9 Analysis of Hedgehog and other signaling pathways in HIO-serosa. Related to FIG. 8. (a): Distribution depicting the percentage of HIOs treated with SU5402, Noggin, DAPT, WNT3A, and SHH that received each score (0-3). (b): Representative images of suspension HIOs treated with Cyclopamine, SAG, IWR1, and CHIR-99021 stained for pCK and WT1. Images depict the most common score(s) for each condition-Cyclopamine: 3, SAG: 2, IWR1: 1, and CHIR-99021:0 (top) and 3 (bottom). Many SAG-treated HIOs formed an inner epithelium with outer serosal mesothelium (top), while others formed a complete serosal mesothelium but did not form an inner epithelium (bottom). (c): Percentage of FACS-isolated HIO-serosa cells that expressed WT1 or αSMA after treatment with SAG or Cyclopamine. HIO-serosa cells were isolated and cultured on Matrigel coated plates in basal media for 24 hours, followed by SAG or Cyclopamine treatment for 6 days before fixation and staining. (d): Representative images of CHIR-99021 treated suspension HIOs stained for ECAD and GATA4, depicting proximal intestinal patterning in HIOs with both score 0 and score 3. (c): Left: Representative images or IWR1 treated suspension HIOs stained for ECAD and KI67. Right: Chart depicting HIO length in IWR1-treated HIOs compared to basal media controls.

FIG. 10. Gene expression in human fetal serosal mesothelium and suspension HIO-serosa. Related to FIG. 4. Table contains all genes enriched in the human fetal serosa and HIO-serosa sub-clusters extracted from scRNA-seq analyses (FIG. 4). Genes highlighted in yellow have a log₂ fold >1.5 in expression in the serosa cluster relative to all other clusters and were thus included in the analysis shown in FIG. 4D.

### DEFINITIONS

As used herein, the term “pluripotent stem cells (PSCs),” also commonly known as PS cells, encompasses any cells that can differentiate into nearly all cells, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of totipotent cells, derived from embryonic stem cells (including embryonic germ cells) or obtained through induction of a non-pluripotent cell, such as an adult somatic cell, by forcing the expression of certain genes.

As used herein, the term “embryonic stem cells (ESCs),” also commonly abbreviated as ES cells, refers to cells that are pluripotent and derived from the inner cell mass of the blastocyst, an early-stage embryo. For purpose of the present invention, the term “ESCs” is used broadly sometimes to encompass the embryonic germ cells as well.

As used herein, the term “induced pluripotent stem cells (iPSCs),” also commonly abbreviated as iPS cells, refers to a type of pluripotent stem cells artificially derived from a normally non-pluripotent cell, such as an adult somatic cell, by inducing a “forced” expression of certain genes.

As used herein, the term “precursor cell” encompasses any cells that can be used in methods described herein, through which one or more precursor cells acquire the ability to renew itself or differentiate into one or more specialized cell types. In some embodiments, a precursor cell is pluripotent or has the capacity to become pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cell and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

As used herein, the term “cellular constituents” are individual genes, proteins, mRNA expressing genes, and/or any other variable cellular component or protein activities such as the degree of protein modification (e.g., phosphorylation), for example, that is typically measured in biological experiments (e.g., by microarray or immunohistochemistry) by those skilled in the art. Significant discoveries relating to the complex networks of biochemical processes underlying living systems, common human diseases, and gene discovery and structure determination can now be attributed to the application of cellular constituent abundance data as part of the research process. Cellular constituent abundance data can help to identify biomarkers, discriminate disease subtypes and identify mechanisms of toxicity.

As used herein, the term “organoid” is used to mean a 3-dimensional growth of mammalian cells in culture that retains characteristics of the tissue in vivo, e.g. prolonged tissue expansion with proliferation, multilineage differentiation, recapitulation of cellular and tissue ultrastructure, etc.

### DETAILED DESCRIPTION OF THE INVENTION

Pluripotent stem cell-derived human intestinal organoids (HIOs) model some aspects of intestinal development and disease, but current culture methods do not fully recapitulate the diverse cell types and complex organization of the human intestine, and are reliant on 3D extracellular matrix or hydrogel systems which limit experimental control and translational potential for regenerative medicine. Experiments described herein describe suspension culture as a simple, low maintenance method for culturing HIOs and for promoting in vitro differentiation of an organized serosal mesothelial layer that is similar to primary human intestinal serosal mesothelium based on single cell RNA-sequencing and histological analysis. Functionally, HIO serosal mesothelium has the capacity to differentiate into smooth muscle-like cells and exhibits fibrinolytic activity. An inhibitor screen identifies Hedgehog and WNT signaling as regulators of human serosal mesothelial differentiation. Collectively, suspension HIOs represent a 3-dimensional model to study the human serosal mesothelium.

Accordingly, the invention disclosed herein generally relates to methods and systems for growing, expanding and/or obtaining human intestinal organoids (HIOs) having a serosal mesothelial layer. In particular, the invention disclosed herein relates to methods and systems for culturing hindgut spheroid tissue in suspension to obtain HIOs having an organized serosal mesothelial layer that is similar to primary human intestinal serosal mesothelium.

In certain embodiments, the present invention provides methods of inducing formation of suspension HIOs having a serosal mesothelial layer comprising obtaining HIOs, and culturing the obtained HIOs in suspension. In some embodiments, the culturing of the obtained HIOs in suspension results in suspension HIOs and in vitro differentiation of an organized serosal mesothelial layer (e.g., suspension HIOs having a serosal mesothelial layer).

Such embodiments are not limited to a particular manner of culturing of the obtained HIOs in suspension. In some embodiments, the culturing of the obtained HIOs in suspension comprises seeding the obtained HIOs with a growth medium. Such embodiments are not limited to a particular type of growth medium. In some embodiments, the growth medium comprises one or more of the following: Advanced Dulbecco's Modified Eagle Medium/Nutrient Mixture F-12 (DMEM/F12) supplemented with 4-(2-hydroxyethyl)-1-piperazineethanesulfonic acid (HEPES), GlutaMAX, Penicillin/Streptomycin, epidermal growth factor (EGF), a BMP inhibiting agent (e.g., Noggin), and R-Spondin. In some embodiments, the HIO growth medium comprises Advanced DMEM/F12 supplemented with HEPES, GlutaMAX, Penicillin/Streptomycin, EGF, Noggin, and R-Spondin.

In some embodiments, seeding the obtained HIOs with the growth medium comprises plating the obtained HIOs in low attachment plates with the growth medium such that the HIOs remain in suspension instead of adhering to the culture dish.

In some embodiments, seeding the obtained HIOs with the growth medium comprises plating the obtained HIOs in low attachment plates with the growth medium having EGF, Noggin, and R-spondin for approximately 72 hours (e.g., 65 hours, 66, 67, 68, 69, 70, 71, 72, 73, 74, 75, 76, 77, 78, 79 hours) followed by culturing with EGF for remaining culture time.

Such methods are not limited to a particular BMP inhibiting agent within the growth medium. In some embodiments, the BMP inhibiting agent is a small molecule or antagonist that inhibits the BMP signaling pathway. In some embodiments, the small molecule or antagonist that inhibits the BMP signaling pathway is Noggin.

In some embodiments, the suspension HIOs having a serosal mesothelial layer comprise an inner ECAD+ epithelium surrounded by VIM+ mesenchyme. In some embodiments, the suspension HIOs having a serosal mesothelial layer comprise expression of one or more of DPP4+ enterocytes, MUC2+ goblet cells, and CHGA+ enteroendocrine cells.

In some embodiments, the serosal mesothelial layer of the suspension HIOs express one or more of WT1, UPK3B, MSLN, and KRT19. In some embodiments, the serosal mesothelial layer of the suspension HIOs express one or more of CAV1, CAV2, and EZR.

In some embodiments, the suspension HIOs having a serosal mesothelial layer comprise an epithelium and/or mesenchyme. In some embodiments, the epithelium expresses one or both of CDX2 and PDX1. In some embodiments, the suspension HIOs having a serosal mesothelial layer comprise epithelium and mesenchyme, wherein the epithelium and mesenchyme express KI67. In some embodiments, the epithelium expresses SOX9.

In some embodiments, the suspension HIOs having a serosal mesothelial layer comprise an apical surface. In some embodiments, the apical surface expresses ZO-1.

In certain embodiments, the present invention provides suspension HIOs having an organized serosal mesothelial layer.

In certain embodiments, the present invention provides methods of treating a mammalian subject having a damaged intestinal tissue with reduced function by engrafting at the site of injury suspension HIOs having an organized serosal mesothelial layer.

In some embodiments, the damaged intestinal tissue includes serosal adhesions occurring following abdominal surgery.

In some embodiments, the methods comprise the steps of administering the suspension HIOs having an organized serosal mesothelial layer to the subject, wherein the suspension HIOs having an organized serosal mesothelial layer engraft at the site of injury and repopulate at least a portion of the site with the suspension HIOs having an organized serosal mesothelial layer, wherein the repopulated suspension HIOs having an organized serosal mesothelial layer supplement the function, thereby treating the subject.

In certain embodiments, the present invention provides methods of treating a mammalian subject having a damaged intestinal tissue with reduced function, comprising:
a) culturing HIOs in suspension, wherein the culturing in suspension results in suspension HIOs and in vitro differentiation of an organized serosal mesothelial layer;
b obtaining suspension HIOs having an organized serosal mesothelial layer;
c) engrafting the obtained suspension HIOs having an organized serosal mesothelial layer at the site of injury, wherein engrafted suspension HIOs having an organized serosal mesothelial layer engraft at the site of injury and repopulate at least a portion of the site with the engrafted cells, wherein the repopulated cells supplement the function, thereby treating the subject.

In some embodiments, the obtained HIOs used in the culturing in suspension embodiments are derived from intestinal hindgut spheroids. In some embodiments, the obtained HIOs used in the culturing in suspension embodiments are pluripotent stem cell-derived human intestinal organoids

In some embodiments, the culturing and obtaining steps are conducted in vitro.

In certain embodiments, the present invention provides compositions comprising suspension HIOs having an organized serosal mesothelial layer produced in vitro from the described methods.

In certain embodiments, the present invention provides kits comprising suspension HIOs having an organized serosal mesothelial layer produced in vitro from the described methods.

In some embodiments, the suspension HIOs having an organized serosal mesothelial layer produced in vitro from the described methods can be used to screen drugs for intestinal tissue uptake and mechanisms of transport. For example, this can be done in a high throughput manner to screen for the most readily absorbed drugs, and can augment Phase 1 clinical trials that are done to study drug intestinal tissue uptake and intestinal tissue toxicity. This includes pericellular and intracellular transport mechanisms of small molecules, peptides, metabolites, salts.

In some embodiments, the suspension HIOs having an organized serosal mesothelial layer produced in vitro from the described methods can be used to identify the molecular basis of normal human intestinal development.

In some embodiments, the suspension HIOs having an organized serosal mesothelial layer produced in vitro from the described methods can be used to identify the molecular basis of congenital defects affecting human intestinal development.

In some embodiments, the suspension HIOs having an organized serosal mesothelial layer produced in vitro from the described methods can be used to correct intestinal related congenital defects caused by genetic mutations. In particular, mutations affecting human intestinal development can be corrected using the suspension HIOs having an organized serosal mesothelial layer produced in vitro from the described methods. In some embodiments, the suspension HIOs having an organized serosal mesothelial layer produced in vitro from the described methods can be used to generate replacement tissue.

In some embodiments, the suspension HIOs having an organized serosal mesothelial layer produced in vitro from the described methods can be used to generate replacement intestinal tissue for intestinal related disorders.

In some embodiments, a diagnostic kit or package is developed to include the suspension HIOs having an organized serosal mesothelial layer produced in vitro from the described methods and based on one or more of the aforementioned utilities.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention. The use of pronouns such as “I”, “we”, and “our”, for example, refer to the inventors.

### Example I

This example demonstrates that HIOs grow and mature in suspension culture.

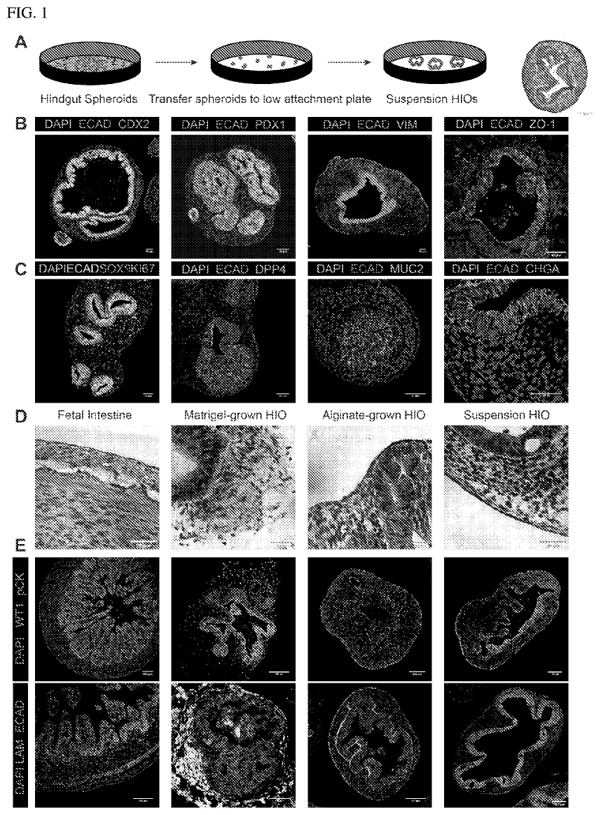

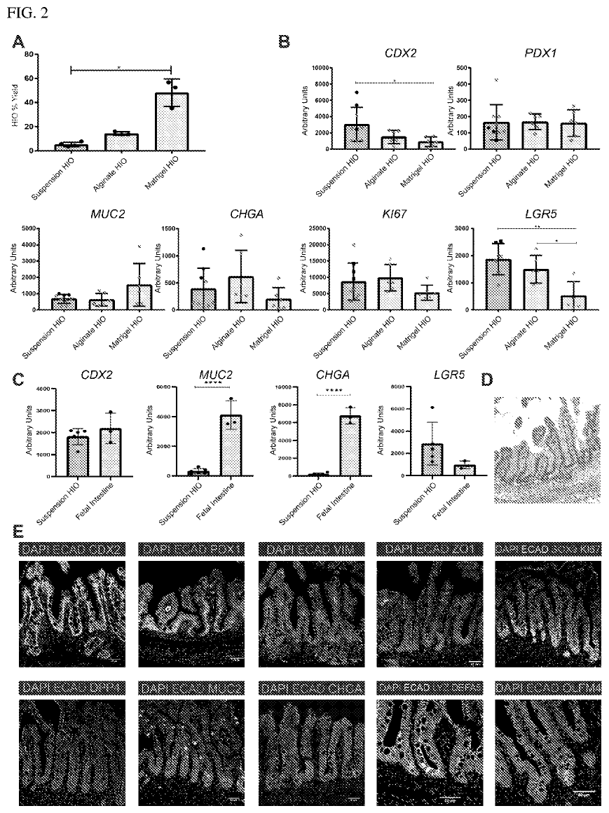

Based on the observation that HIOs can be cultured in unmodified alginate, a bioinert hydrogel that provides purely mechanical support (Capeling et al., 2019), it was hypothesized that the 3D support provided by ECM may be dispensable for HIO development. This hypothesis was tested using suspension culture as an alternative method to grow HIOs. Suspension culture has been utilized in other organoid systems (Hohwieler et al., 2017; Kumar et al., 2019; Takahashi et al., 2018) and is a desirable culture method due to its simplicity, cost-effectiveness, and amenability to scale-up. Intestinal hindgut spheroids were generated using previously described directed differentiation methods (Capeling et al., 2020; Finkbeiner et al., 2015; McCracken et al., 2011; Spence et al., 2011). Instead of transferring spheroids to a 3D droplet of alginate or Matrigel, spheroids were transferred to a low attachment plate containing HIO growth media (FIG. 1A). By 4 weeks of culture, suspension HIOs resembled alginate and Matrigel HIOs as assessed by bright field microcopy and hematoxylin and eosin (H&E) staining, with a defined inner epithelium and outer mesenchyme (FIG. 1A, B).

Histological techniques were used to examine the effects of suspension culture. HIOs cultured in suspension developed an inner ECAD+epithelium surrounded by VIM+mesenchyme. The epithelium of suspension HIOs expressed the intestinal transcription factor CDX2 as well as the duodenum marker PDX1. Additionally, suspension HIOs had a properly polarized epithelium as the tight junction marker ZO-1 was expressed across the apical surface (FIG. 1C). Suspension HIOs expressed the proliferation marker KI67 throughout the epithelium and mesenchyme, demonstrating that HIOs are able to proliferate in suspension. Similar to alginate and Matrigel HIOs cultured in vitro, the epithelium of suspension HIOs was largely immature as evidenced by broad expression of the progenitor marker SOX9 (Hill et al., 2017). However, suspension HIOs gave rise to differentiated intestinal epithelial cell types including DPP4+ enterocytes, MUC2+ goblet cells, and CHGA+ enteroendocrine cells (FIG. 1D) (Capeling et al., 2019). To directly compare suspension HIOs to alginate and Matrigel HIOs, qRT-PCR was performed and it was found that expression levels of most markers tested displayed no significant differences between conditions (FIG. 2B). While HIOs are relatively immature in vitro (Finkbeiner et al., 2015; Yu et al., 2021), it was found that suspension HIOs undergo maturation when transplanted into mice, as has been described for alginate and Matrigel HIOs (Capeling et al., 2019; Watson et al., 2014) (FIG. 2B-E). Together, these results demonstrate that suspension culture supports the development of HIOs and gives rise to an epithelium that resembles alginate and Matrigel HIOs in vitro and in vivo.

### Example II

This example demonstrates that HIOs cultured in non-adherent conditions form putative serosal mesothelium.

While suspension HIO epithelium was similar to alginate and Matrigel HIOs, differences were observed in mesenchymal organization. In alginate and suspension HIOs, the mesenchyme became radially oriented in a manner that more closely resembled the fetal intestinal mesenchyme. This may be due to the non-adherent nature of alginate and suspension culture, since mesenchymal cells were unable to interact with a surrounding matrix and spread away from the epithelium as in Matrigel. In order to characterize mesenchymal differences across culture conditions, H&E staining was performed. Notably, an outer cell layer in alginate and suspension HIOs were observed that resembled the serosal mesothelium of the developing human intestine (Bloom and Fawcett, 1975; Mutsaers, 2002), while a defined outer layer was not observed in Matrigel HIOs (FIG. 1D).

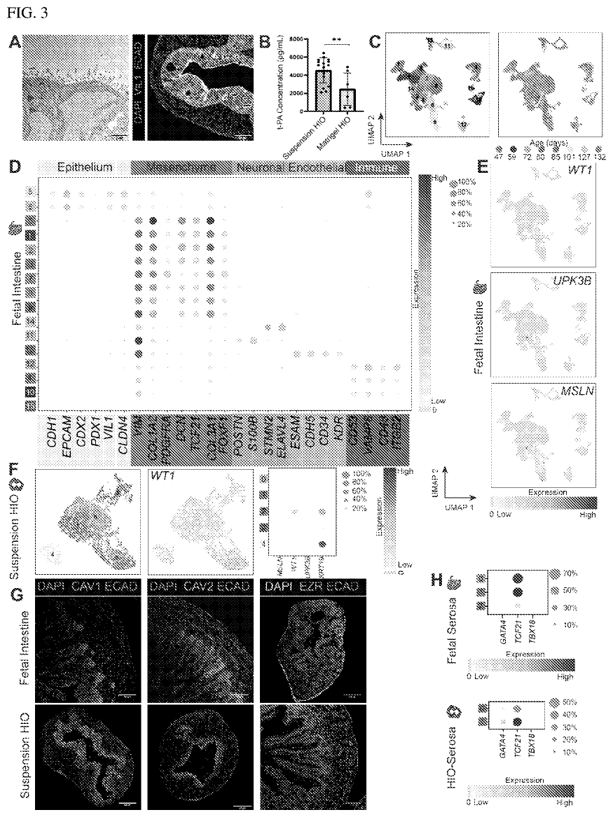

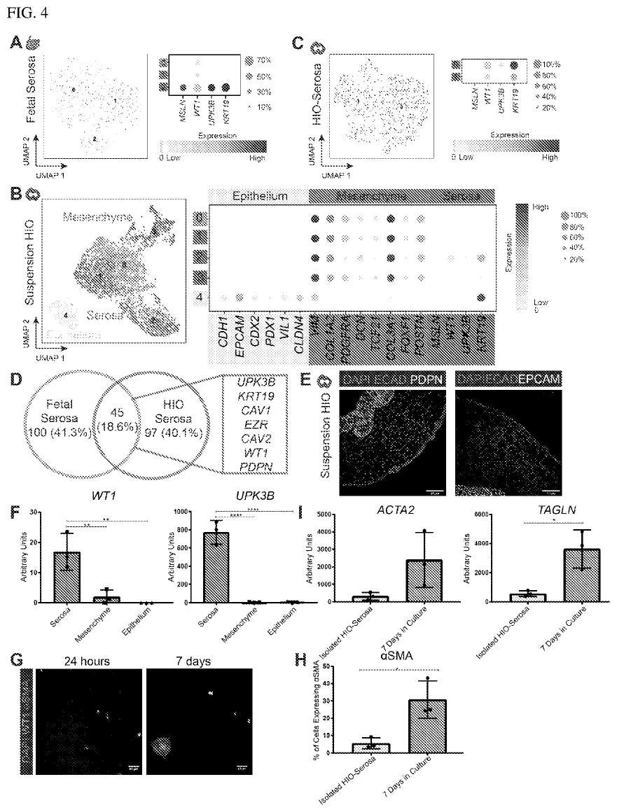

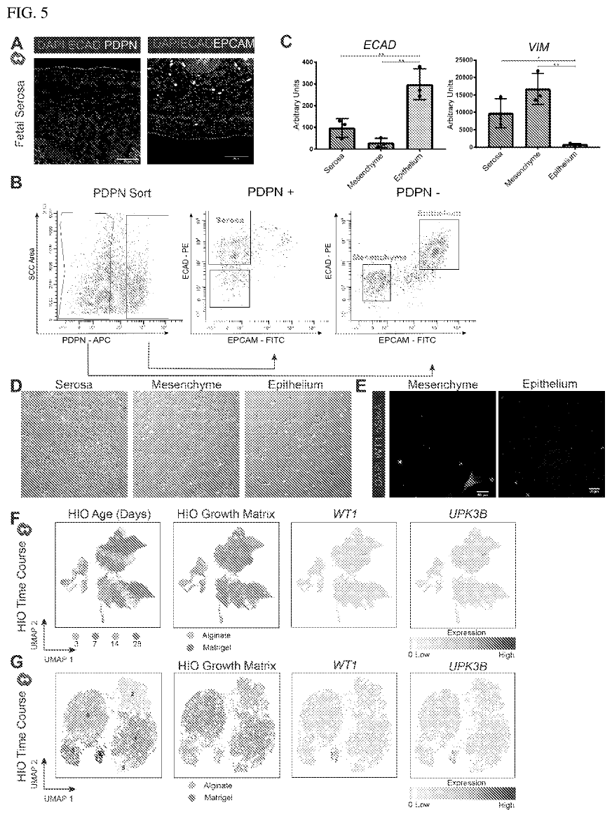

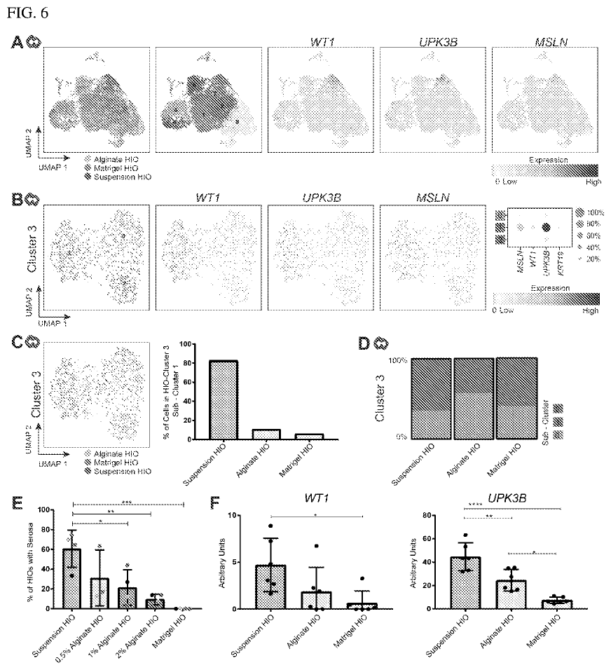

In order to confirm serosal identity in alginate and suspension HIOs, immunostaining for mesothelial markers was performed. Wilms' tumor protein (WT1) and Cytokeratin proteins are well characterized markers of mesothelial cells (Chan et al., 1988; Foley-Comer et al., 2002; Kumar-Singh et al., 1997; Watt et al., 2004; Wilm et al., 2005; Winters and Bader, 2013). Additionally, previous studies have indicated that the serosal mesothelium sits on a laminin-rich basement membrane (Mutsaers, 2002; Winters and Bader, 2013). In Matrigel HIOs, a serosal mesothelium was never observed as WT1 was disperse throughout the mesenchyme, and Matrigel HIOs did not form an outer basement membrane (FIG. 1E). In both alginate and suspension HIOs, \ a putative serosal mesothelium marked by co-expression of WT1 and pCK, and lined by a LAM+ basement membrane was observed (FIG. 1E). Mesothelial staining patterns in alginate and suspension HIOs resembled the serosal mesothelium of the human fetal intestine. Additionally, suspension HIOs formed a microvillus-lined surface typical of mesothelial cells based on TEM and immunostaining for the microvillus marker VIL1 (FIG. 3A) (Mutsaers, 2004). Levels of the secreted fibrinolytic agent, t-PA, which mediates fibrinolytic activity of the serosa, were assessed and it was found that suspension HIOs had higher levels of t-PA secreted into the media than Matrigel HIOs (FIG. 3B). This suggests that HIO-serosa exhibits some expected functions of human serosal mesothelium, including fibrinolytic activity and microvillus formation (Mutsaers, 2004; Ziprin et al., 2003).

### Example III

This example demonstrates that HIO-serosa resembles human serosa at the molecular level.

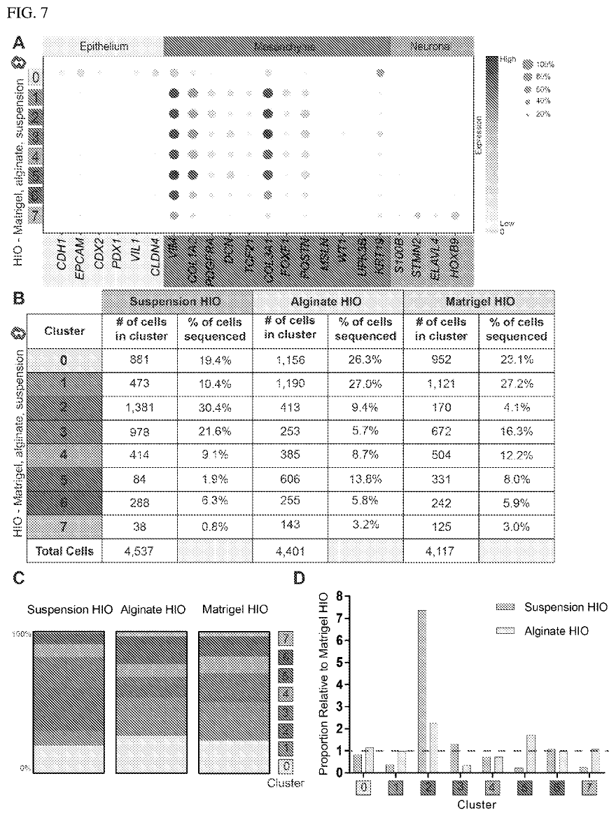

To characterize the developing human serosa and more closely compare HIO-serosa to human fetal serosa, published single cell RNA-sequencing (scRNA-seq) human fetal intestine data was analyzed and scRNA-seq data generated for suspension HIOs (Holloway et al., 2021; Holloway et al., 2020) (FIG. S2C, D). From the human fetal data, a small subset of cells within mesenchymal cluster 8 that expressed mesothelial markers WT1 (Rudat et al., 2014), UPK3B (Kanamori-Katayama et al., 2011), MSLN (Rinkevich et al., 2012), and KRT19 (Lua et al., 2015) was identified (FIG. 3E). In order to better define the subset of human fetal serosal cells, 761 cells from cluster 8 were computationally extracted and re-clustered to identify a subset of cells (sub-cluster 2) as human serosal mesothelium based on expression of WT1, UPK3B, MSLN, and KRT19 (FIG. 4A). scRNA-seq on suspension HIOs was similarly performed, which displayed expected epithelial (cluster 4) and mesenchymal (clusters 0, 1, 2, 3) cell lineages (FIG. 4B). Cells within cluster 2 that expressed mesothelial markers were identified (FIG. 4B, 3F). Cluster 2 was computationally extracted and sub-clustering on 989 cells was performed to identify sub-cluster 0 as a serosa-like population expressing the highest levels of WT1, UPK3B, MSLN, and KRT19 (FIG. 4C).

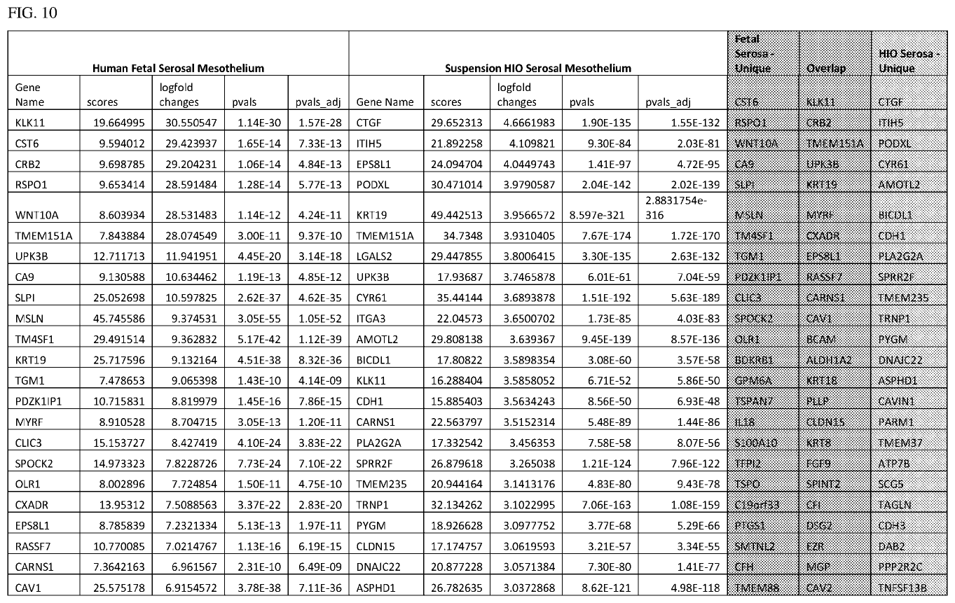

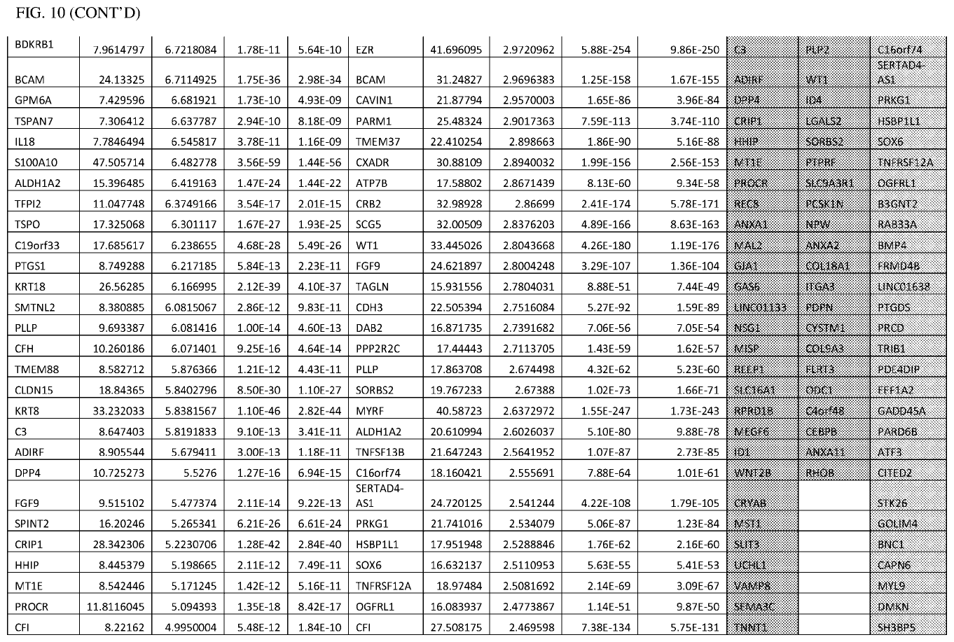

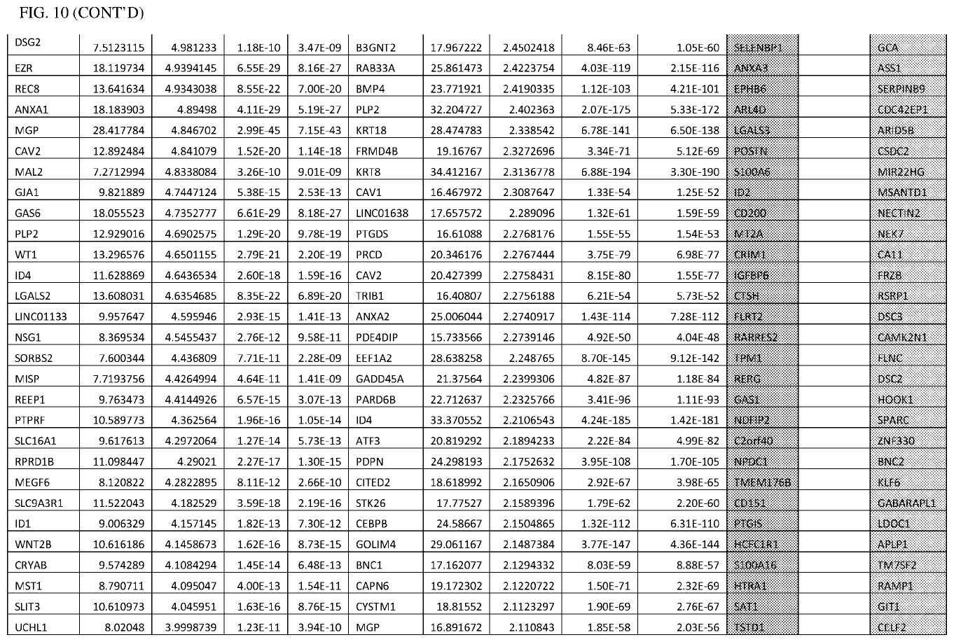

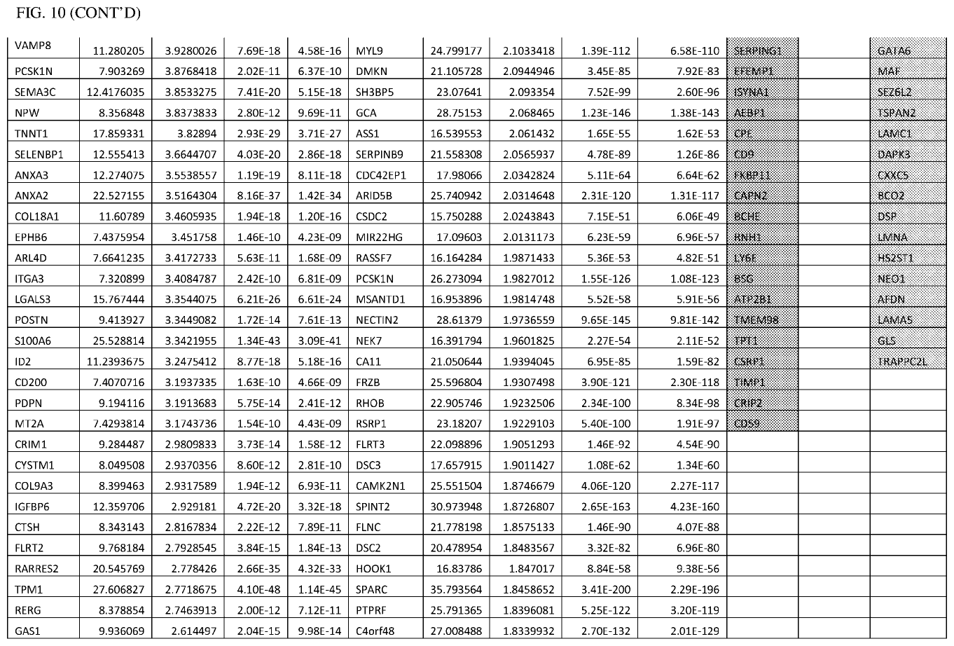

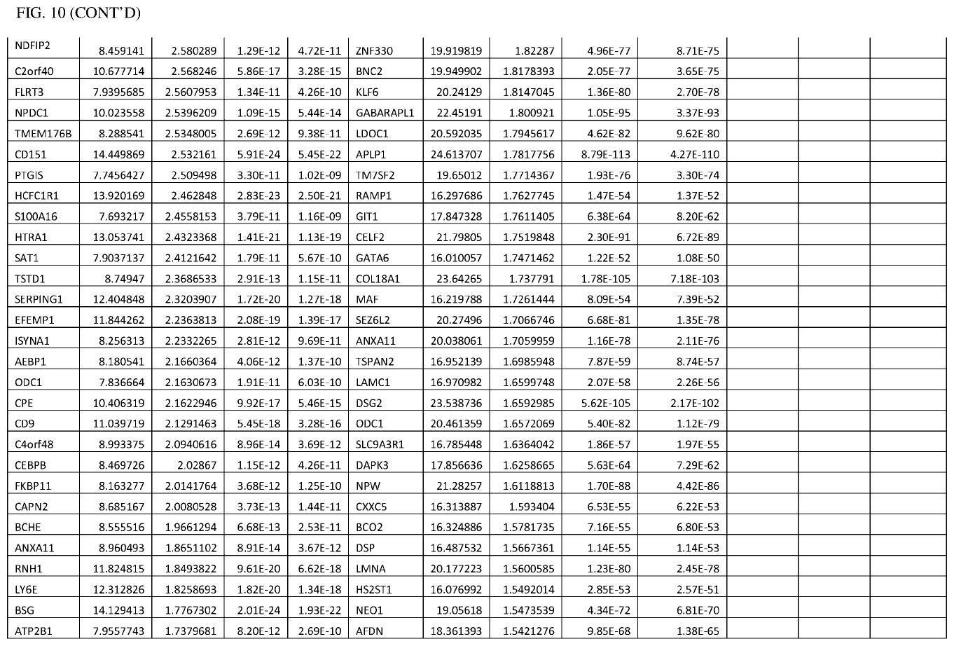

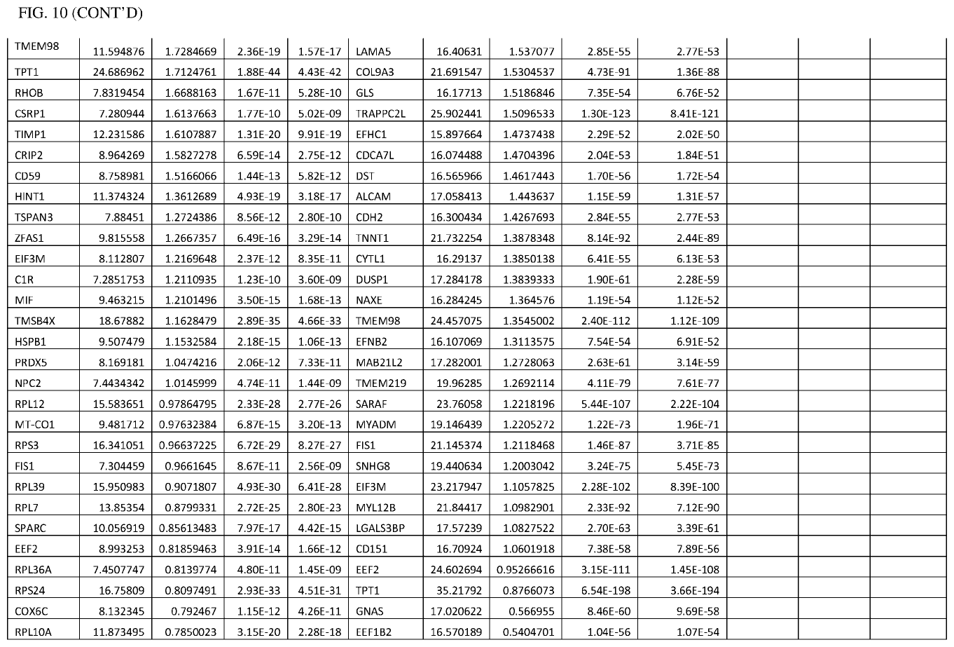

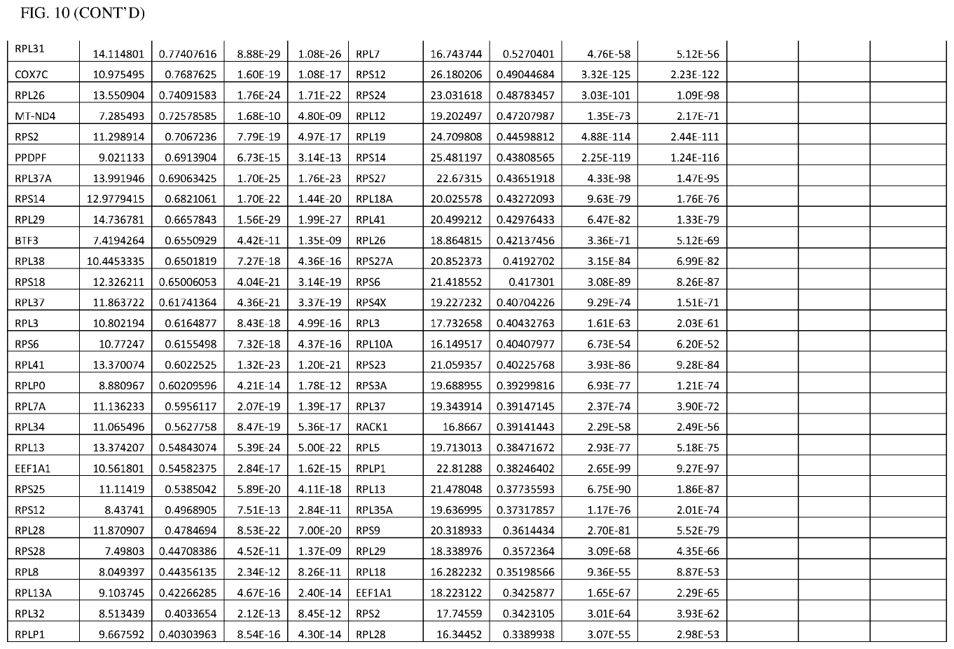

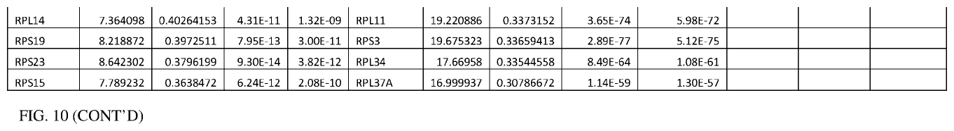

From the extracted serosa sub-clusters within human intestine and suspension HIOs, lists of the most differentially expressed genes defined as a log₂ fold >1.5 in expression of a gene in the serosa cluster relative to all other clusters was identified (FIG. 10). The human fetal and HIO-serosa shared 45 genes in common: 18.6% of all genes or 31.0% and 31.6% of genes in each list, respectively—a statistically significant overlap (FIG. 4D). When comparing any cluster to all other clusters (fetal intestine, suspension HIO, fetal serosa, HIO-serosa), the average overlap between gene sets is 2.4%. This suggests that comparing in vivo to in vitro serosa has a 7.8-fold increase in gene expression overlap than would be expected by chance. The list of genes upregulated in both human and HIO-serosa revealed well-documented mesothelial genes and markers that are not well defined in the literature including CAV1, CAV2, and EZR (FIG. 4D). Histological analysis confirms that these markers are expressed in human fetal and HIO-serosa (FIG. 3G). This analysis provides further confirmation that HIO-serosa resembles human intestinal serosa and identifies previously under-studied genes that mark this population.

### Example IV

This example demonstrates that HIO-serosa retains functional capability to differentiate into smooth muscle-like cells.

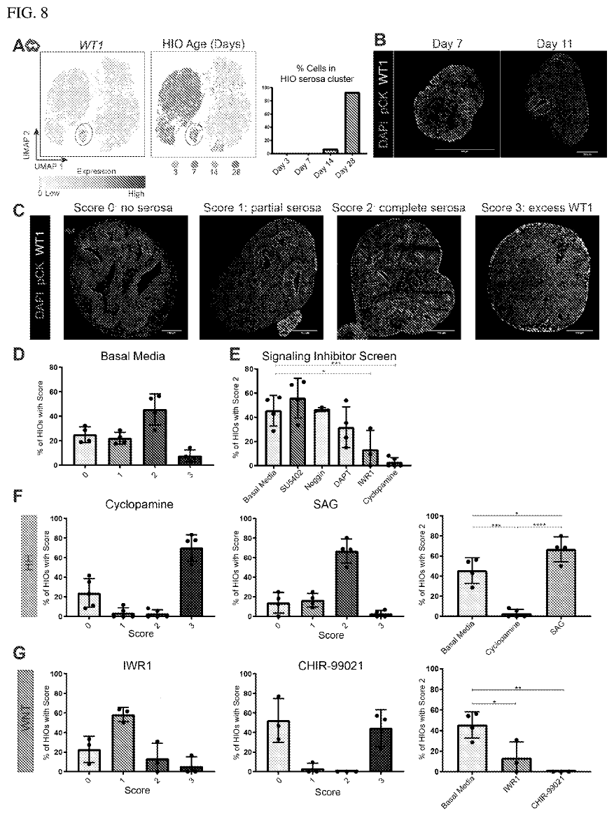

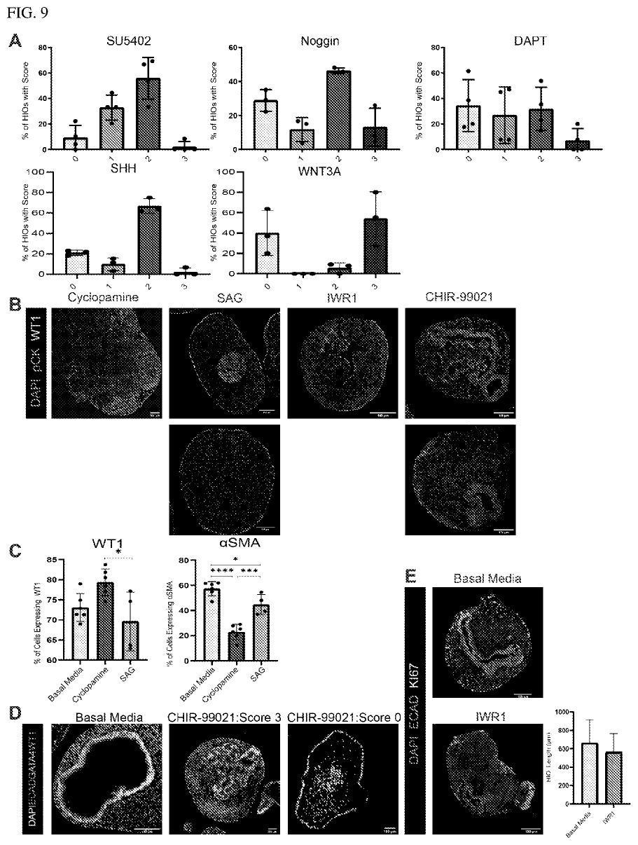

Mesothelial cells have a well-described function of giving rise to mesenchymal cells including vascular smooth muscle (Carmona et al., 2013; Que et al., 2008; Wilm et al., 2005). Additionally, previous studies have demonstrated that isolated mesothelial cells undergo differentiation into smooth muscle-like cells in vitro (Kawaguchi et al., 2007; Wada et al., 2003). In order to test if HIO-serosa exhibits the expected functionality of differentiating into smooth muscle-like cells, a FACS strategy was devised to enrich serosal cells. It was observed that human and HIO-serosa express PDPN and ECAD but not EPCAM (FIG. 4E, 5A). This allowed separation of serosa (PDPN+/ECAD+/EPCAM−) from epithelium (PDPN−/ECAD+/EPCAM+) and non-serosal mesenchyme (PDPN−/ECAD−/EPCAM−) (FIG. 5B). qRT-PCR was performed on the sorted cell populations and it was verified that serosal mesothelial genes including WT1 and UPK3B were significantly enriched in the sorted PDPN+/ECAD+/EPCAM− population (FIG. 4F, 5C).

Sorted HIO-serosa cells were cultured in vitro for 24 hours as an initial time point (Isolated HIO-Serosa) and compared to sorted cells cultured for 7 days. A significant increase in the percentage of cells that were αSMA+ after 7 days was observed compared to the 24-hour time point, as 30.8±6% of cells expressed αSMA after 7 days (FIG. 4G, H). qRT-PCR was additionally performed to compare freshly sorted HIO-serosa to HIO-serosa cells cultured in vitro for 7 days (FIG. 4H) and observed increased expression of smooth muscle markers ACTA2 and TAGLN (FIG. 4I). These results demonstrate that HIO-serosa behaves in a manner typical of mesothelial cells differentiating into smooth-muscle like cells.

### Example V

This example demonstrates that HIO-serosa formation is enhanced in suspension culture.

The data (Examples I-IV) suggested that suspension and alginate HIOs possess an organized serosa-like layer whereas Matrigel HIOs sometimes possess few disorganized WT1+ cells. In order to further interrogate similarities and differences, scRNA-seq analyses of 28-day HIOs cultured in Matrigel, alginate, and suspension were combined. We observed that cells from all 3 conditions were represented in every UMAP cluster (FIG. 6A). The proportion of cells that were epithelial was similar between culture conditions, while mesenchymal populations contributed different proportions (FIG. 6A, 7B-D). From this combined analysis, a population within cluster 3 was identified that expressed mesothelial markers (FIG. 6A). Experiments were conducted that computationally extracted and re-clustered this population, revealing that sub-cluster 1 was enriched for WT1, UPK3B, MSLN, and KRT19 (FIG. 6B). Within sub-cluster 1, 82.8% of cells originated from suspension HIOs, with 11.0% and 6.2% from alginate and Matrigel HIOs, respectively (FIG. 6C). Experiments were conducted that additionally compared the distribution of cells within each sub-cluster across conditions and found that 61.5% of suspension HIO cells in cluster 3 fell into the mesothelial sub-cluster 1, compared with 31.6% and 6.7% in alginate and Matrigel, respectively (FIG. 6D). This analysis suggests that serosa formation is enhanced in suspension culture.

To further quantitate HIO-serosa formation across conditions, the percentage of HIOs cultured in suspension, alginate, and Matrigel that formed a serosa were calculated. The highest frequency of serosa formation was observed in suspension culture, and it was observed that serosa formation decreased as alginate density increased (FIG. 6E). Similarly, high expression of the mesothelial markers WT1 and UPK3B was observed by qRT-PCR in suspension culture with decreased expression in alginate HIOs and little to no expression in Matrigel HIOs (FIG. 6F). If serosa formation was driven solely by lack of adhesion, we would expect to see no differences based on alginate gels of any concentration, given that alginate gels of increasing polymer concentration exhibit a higher storage modulus (Capeling et al., 2019). These data suggest that compressive or attractive forces from the ECM may hinder mesothelial development, while the absence of compressive forces in suspension culture promotes organization of a serosal mesothelium.

### Example VI

This example demonstrates that Hedgehog and WNT signaling are implicated in serosal mesothelial development.

Based on similarities between human and HIO-serosa, we utilized suspension HIOs to investigate signaling pathways involved in serosa formation, since little is known about mesothelial cell differentiation in the human intestine (Thomason et al., 2012; Winters et al., 2012). Prior to interrogating signaling pathways involved in serosa formation, we determined when serosa first differentiates by analyzing an scRNA-seq time-course on HIOs (Holloway et al., 2020) as well as immunofluorescent staining (FIG. 4A,B, S3F). We found that HIO-serosa formation begins after day 7 of culture and is complete between day 14-28. Using this developmental timeline, we applied activators/inhibitors of major signaling pathways known to be involved in mesothelial differentiation or intestinal mesoderm development (Colunga et al., 2019; Dixit et al., 2013; Inagaki et al., 2019; Kolterud et al., 2009; Kruithof et al., 2006; Schlueter et al., 2006; Tian et al., 2015; VanDussen et al., 2012; Verzi and Shivdasani, 2008) using suspension HIOs, and assessed their effects on the serosa.

Suspension HIOs were grown for 7 days before applying signaling activators/inhibitors to the culture medium so as not to interfere with early developmental patterning. Starting on day 8, we treated suspension HIOs with inhibitors of the FGF (SU5402), BMP (Noggin), Notch (DAPT), WNT (IWR1), and HH (Cyclopamine) signaling pathways. To assess serosa differentiation, we devised a qualitative grading scheme to score HIOs based on staining for WT1 and pCK. Scoring ranged from 0 to 3, where 0 indicated no serosa, 1 indicated a partial WT1+pCK+ outer serosa, 2 indicated a complete or ‘perfect’ serosa with WT1+pCK+ staining surrounding the HIO, and 3 indicated a full or partial outer serosa with additional ectopic WT1 staining in the mesenchyme (FIG. 8C).

45.5±6% of HIOs in basal media were scored ‘2’, a complete serosa (FIG. 8D). We compared the percentage of HIOs with score 2 in basal media to inhibitor-treated HIOs to determine if blocking key signaling pathways led to a change in the frequency of HIO-serosa formation. Inhibiting FGF, BMP, or Notch signaling did not significantly alter the percentage of HIOs that formed a complete serosa (FIG. 8E, 9A). On the other hand, inhibition of WNT or HH signaling with IWR1 or Cyclopamine, respectively, led to a significant decrease in the percentage of HIOs with score 2 (FIG. 8E).

To follow up on these findings, we pursued HH and WNT modulation experiments. Inhibition of HH signaling with Cyclopamine in suspension HIOs led to a majority of HIOs with score 3, indicating that HH inhibition led to excess WT1 expression throughout the mesenchyme, while activation of HH signaling with SAG resulted in a majority of HIOs scoring 2 (FIG. 8F, 9B). Cyclopamine treatment led to a significant decrease in the percentage of HIOs with score 2 compared to control or SAG treatment, while SAG-treated HIOs had a significantly higher proportion of score 2 compared to controls (FIG. 4F). Thus, HH signaling may be necessary to restrict WT1 localization to the outer cell layer in the developing intestine.

In order to determine if HH modulation directly affects the intestinal serosa, we FACS isolated serosal mesothelial cells from suspension HIOs and treated these cells with SAG and Cyclopamine. After 7 days, we used immunofluorescence to stain for WT1 and αSMA, and calculated the percentage of cells in each condition that expressed each marker. We found that the percentage of αSMA+ cells in the Cyclopamine group was significantly lower than the percentage of αSMA+ cells in both the basal media and SAG groups (FIG. 9C), suggesting that HH inhibition may block the ability of mesothelial cells to differentiate into αSMA+mesenchymal cells. This is consistent with observations in the mesothelium of the developing mouse lung (Dixit et al., 2013).

Inhibition of WNT signaling with IWR1 resulted in a majority of HIOs with a score of 1 (FIG. 8G, 9B), suggesting that endogenous WNT signaling may be required for proper serosal mesothelial formation. Stimulation of WNT signaling with CHIR-99021 caused aberrant serosa formation and resulted in HIOs with score 0 or 3 (FIG. 8G, 9B), suggesting that altered WNT signaling disrupts serosa formation. IWR1 treatment did not significantly alter HIO length, suggesting that WNT inhibition stunts mesothelial development without limiting the overall growth of suspension HIOs. Together, these results suggest that HH and WNT signaling are necessary for proper differentiation and patterning of the human intestinal serosal mesothelium.

### Example VII

This example provides a discussion related to Examples I-VI.

In Examples I-VI we described suspension culture as an alternative to hydrogel or Matrigel culture for human intestinal organoids. Suspension culture provides an advantage over Matrigel by removing biological variability and reducing cost. Strikingly, HIOs cultured in non-adherent alginate or suspension formed a serosal mesothelium that resembled that of the human fetal intestine. scRNA-seq analysis of HIOs revealed a serosa population that was significantly similar to the human serosa cluster. We hypothesize that non-adherent culture conditions (alginate, suspension) promote mesothelial development compared to Matrigel as HIO mesenchymal cells are able to self-organize rather than migrating away from the epithelium. There were some differences in gene expression between human and HIO serosal mesothelium, which can be attributed to differences between the in vivo environment and in vitro culture (Miller et al., 2020). Suspension culture enhances HIO-serosa formation compared to alginate-grown HIOs which suggests that lack of a compressive environment may enable mesothelial differentiation and organization. The emergence of a serosal mesothelium in suspension may be due to the fact that suspension culture mimics early developmental events in vivo in which the gut tube is essentially suspended in fluid (Pansky, 1982).

In order to evaluate the functionality of HIO-serosa, we devised a method to FACS-sort mesothelial cells from suspension HIOs and confirmed that they undergo expected differentiation into smooth muscle-like cells in vitro. Thus, HIOs are a promising model system to study mesothelial development and differentiation within a human model system in vitro. The ability to culture and sort mesothelial cells from HIOs may prompt further studies into factors driving mesothelial to mesenchymal differentiation. Additionally, isolated HIO-serosa may be a promising source of mesothelial cells for therapeutic approaches (Colunga et al., 2019; Herrick and Mutsaers, 2007; Lachaud et al., 2015).

HIO-serosa serves as an in vitro model system to study how the serosa originates from mesodermal progenitors in the developing human intestine. Contrary to reports on the mesothelium of the heart (Kruithof et al., 2006), we did not find mesothelial differentiation to be dependent upon BMP or FGF. This may highlight organ or species-specific differences in mesothelial development. We demonstrated that inhibition of WNT or HH signaling disrupted serosa formation in suspension HIOs. However, the precise mechanism by which WNT and HH play a role in serosal development, including whether these pathways have a direct vs. indirect effect, is still unclear. FACS isolated HIO-serosal cells treated with Cyclopamine do not have significantly increased WT1 expression, but rather exhibit lower levels of αSMA expression compared to controls (FIG. 9C), while whole HIOs treated with Cyclopamine exhibit excess ectopic WT1 expression in the mesenchyme (FIG. 8F, 9C). This data suggests that HH is not directly inducing or blocking serosa differentiation, but may function to limit the ability of serosa to differentiate into other cell types such as αSMA+ mesenchyme. Based on these results, we propose a model where blocking HH inhibits the ability of WT1+ serosal cells to differentiate into non-serosal cell types such as smooth muscle-like cells.

### Example VIII

This example describes the material and methods utilized in the experiments described in Examples I-VII.

### Data and Code Availability:

Sequencing data used in the experiments described herein is deposited at EMBL-EBI ArrayExpress. Single-cell RNA sequencing of human tissue: human fetal intestine (ArrayExpress: E-MTAB-9489) (Holloway et al., 2021); human fetal intestine (E-MTAB-9363) (Holloway et al., 2020); human fetal intestine (deposition of data to ArrayExpress is in progress)—this study; HIO (ArrayExpress: E-MTAB-9228) (Holloway et al., 2020); HIO (deposition of data to ArrayExpress is in progress)—this study. Accession numbers for deposited data are also provided in the Key Resources Table. Any additional information required to reanalyze the data reported in this work is available from the Lead Contact upon request.

hESC/hIPSC Lines and Generation of hPSC-Derived Intestinal Organoids

This study includes data from HIOs generated across 4 hPSC lines: Human ES lines H9 (NIH registry #0062, RRID: CVCL_9773, female) and UM63-1 (NIH registry #0277, RRID: CVCL_R782), as well as human iPSC lines WTC11 (RRID: CVCL_Y803, male) and 72.3 (McCracken et al., 2014). All experiments using hPSCs were approved by the University of Michigan Human Pluripotent Stem Cell Research Oversight Committee. hPSC lines and HIOs are routinely monitored for mycoplasma using the MycoAlert Mycoplasma Detection Kit (Lonza). hPSC lines are routinely karyotyped and H9 cells were authenticated using Short Tandem Repeat (STR) DNA profiling (Matsuo et al., 1999) at the University of Michigan DNA Sequencing Core and were found to exhibit an STR profile identical to previously described characteristics (Josephson et al., 2006).

Stem cell maintenance and differentiation into HIOs was carried out as recently described in detail (Capeling et al., 2019; Capeling et al., 2020; Hill et al., 2017; McCracken et al., 2011; Tsai et al., 2017). Cell culture was carried out in a 37 °C tissue culture incubator. hPSCS were maintained in mTeSR 1 or mTeSR Plus cultured media (Stemcell Tecnologies) and enzymatically passaged with dispase (Gibco). hPSCs underwent directed differentiation into definitive endoderm over a 3-day treatment with Activin A (100 ng/mL, R & D Systems) added into RPMI media supplemented with 0%, 0.2%, 2% HyClone dFBS on subsequent days. Endoderm was differentiated into hindgut by treatment with FGF4 (500 ng/ml (Sugawara et al., 2014)) and CHIR99021 (2 μM, APExBIO).

Mid/Hindgut spheroids that budded off from the monolayer during differentiation were collected after days 5 and 6 of hindgut induction. Spheroids were embedded in alginate or Matrigel as previously described (Capeling et al., 2019), or transferred to low attachment plates for suspension culture to enable growth into HIOs. Organoids were maintained in basal growth media consisting of Advanced DMEM/F12 with 1× B27 (Thermo Fisher), GlutaMAX (Gibco, 1×), penicillin-streptomycin (Gibco, 100 U ml⁻¹ penicillin; 100 μg ml⁻¹ streptomycin), and HEPES buffer (Gibco, 15 mM). Organoid basal growth media was supplemented with epidermal growth factor (EGF) (R&D Systems; 100 ng/ml), Noggin-Fc (100 ng/mL) (purified from conditioned media (Heijmans et al., 2013)), and R-Spondin1 (5% conditioned medium (Ootani et al., 2009)) for the first three days of culture to promote patterning into proximal small intestine. On the third day after embedding, media was changed to basal growth media supplemented with EGF alone. HIOs were maintained in EGF-supplemented media for the duration of culture. Media was changed every 5-7 days. Organoids were not passaged to avoid disrupting the serosal mesothelium.

Experiments involving pooled sets of HIOs involved n>10 organoids per experiment. See Figure legends for more information on sample size for each experiment. For experimental treatment groups including the HIO inhibitor screen, equal numbers of HIOs were randomly allocated to each experimental group.

### Human Tissue

Normal, de-identified human fetal intestinal tissue was obtained from the University of Washington Laboratory of Developmental Biology and shipped overnight in Belzer-UW Cold Storage Solution (ThermoFisher, NC0952695) with cold packs as previously described (Miller et al., 2020). All research utilizing human tissue was approved by the University of Michigan institutional review board. This study includes male and female samples from day 47 to day 132 post-conception.

### Generation of Low-Attachment Plates and Suspension Culture

Low attachment plates were generated using previously published methods (Choi et al., 2011). In summary, poly(2-hydroxyethyl methacrylate) (pHEMA) coating solution was prepared by dissolving 4 g of pHEMA (Thermo Fisher) into 40 mL of 95% ethanol with 10 mM NaOH. The solution was shaken immediately upon addition of pHEMA to avoid precipitation and rotated continuously overnight until fully dissolved. 1 mL of pHEMA coating solution was applied to each well of a 6 well plate inside a biosafety cabinet to generate a low attachment culture plate. The plate was rocked side to side to ensure distribution of pHEMA, and then pHEMA was collected for re-use. The low attachment plate was left in the biosafety cabinet overnight with the plate lid open to allow evaporation of excess coating solution, and UV was turned on for 15 minutes to sterilize the plate. Low attachment plates were rinsed twice with 1×PBS prior to addition of spheroids to remove excess pHEMA. Approximately 200 spheroids were transferred to 1 well of a 6-well low-attachment plate containing 5 mL of media for suspension HIO culture. To change media without aspirating organoids in suspension, media was collected under a stereomicroscope using a P1000 after allowing organoids to settle to the bottom of the plate.

### Mouse Kidney Capsule Transplantation

The University of Michigan and Cincinnati Children's Hospital Institutional Animal Care and Use Committees approved all animal research. HIOs were cultured for 4 weeks in suspension and then collected for transplantation, at which point HIOs were implanted under the kidney capsules of immunocompromised NOD-scid IL2Rg-null (NSG) mice (Jackson Laboratory strain no. 0005557) as previously described (Finkbeiner et al., 2015; Watson et al., 2014). In summary, mice were anaesthetized using 2% isoflurane. A left-flank incision was used to expose the kidney after shaving and sterilization with isopropyl alcohol. HIOs cultured in suspension were surgically implanted beneath mouse kidney capsules using forceps. Prior to closure, an intraperitoneal flush of Zosyn (100 mg kg-1; Pfizer) was administered. Mice were euthanized for retrieval of tHIOs after 8 weeks. Results shown are representative of one experiment performed with a total of n=6 mice, with at least one organoid implanted per kidney capsule depending on HIO size.

### Flow Cytometric Analysis of HIOs

Suspension HIOs were transferred to a Petri dish containing TrypLE Express (Thermo Fisher) and mechanically cut into small pieces using a scalpel. TrypLE and dissociated HIOs were then transferred to a 15 mL conical tube and placed into a tissue culture incubator at 37 °C. HIOs were digested in TrypLE at 37 °C until the tissue was fully dissociated (~1.5-2 hours), and tissue was agitated roughly every 15 minutes during digestion by vortexing and pipetting up and down with a P1000. Once digestion was complete, reactions were quenched by adding a 2× volume of DMEM: F12 media. HIO suspensions were passed through a 70 μm filter and then centrifuged at 300 g for 5 min at 4 °C. Cells were then rinsed in staining buffer (1×PBS, 2% BSA, 1×PenStrep, 10 mM Y-27632 (Reagents Direct)), centrifuged, and re-suspended in an appropriate volume of staining buffer for antibody staining (100-200 μl). Cell suspensions were stained with conjugated FACS antibodies (Key Resources Table) and DAPI (0.2 μg/ml) at 4 °C for 30 minutes. Cells were then rinsed with 3 mL of staining buffer, centrifuged, and re-suspended in 500 μL staining buffer. Flow cytometric analysis was performed using a Sony SY3200 cell sorter and accompanying software.

Cells were first gated on PDPN, and then passed through a secondary gate on ECAD and EPCAM. From the PDPN+ population, a PDPN+/ECAD+/EPCAM− population was collected as HIO serosal mesothelium. From the PDPN− population, a PDPN−/ECAD+/EPCAM+ population was collected as HIO epithelium, and a PDPN−/ECAD−/EPCAM− population was collected as HIO mesenchyme.

### Culture of Isolated HIO-Serosa

Following FACS isolation, PDPN+/ECAD+/EPCAM-serosa was plated on Matrigel-coated 24-well plates in basal organoid growth media supplemented with 10 mM Y-27632 (Reagents Direct) and 0.4 μg/mL Hydrocortisone (Sigma-Aldrich) (Yung et al., 2006). Y-27632 was removed after 24 hours for 7-day cultures. For HH modulation experiments, Cyclopamine (5 μM) or SAG (2 μM) was added to basal organoid growth media with 0.4 μg/mL Hydrocortisone for 6 days following the 24-hour culture in basal media.

### HIO-Serosa Signaling Screen

Hindgut spheroids were collected and cultured in suspension with basal organoid growth medium supplemented with (EGF) (100 ng/mL), Noggin-Fc (100 ng/mL), and R-Spondin2 (5% conditioned medium) for 3 days. On the third day after collection, media was changed to basal organoid growth medium supplemented with only EGF (100 ng/mL). After 7 days, signaling activators or inhibitors were applied by changing cultured medium to organoid growth medium supplemented with EGF (100 ng/mL-basal medium control condition) and signaling compounds, including DAPT (10 μM ng/ml), IWR1 (10 μM), CHIR99021 (2 μM), Cyclopamine (5 μM), SAG (2 μM), SU5402 (10 μM), Noggin-Fc (100 ng/mL), BMP4 (100 ng/mL), SHH (33.3 ng/mL), and WNT3A (250 ng/mL). HIOs were then cultured in medium containing signaling compounds for 3 weeks (28 days total in culture). After 28 days, HIOs were collected and fixed for immunofluorescence staining or flash-frozen for qRT-PCR analysis.

To score HIOs and determine effects of signaling compounds on serosal mesothelium, an entire batch of matched HIOs was fixed and stained for WT1 and pCK. Each HIO in one plane of section was given a score between 0 and 3 based on WT1 and pCK immunofluorescent staining. Scoring was as follows-0: no WT1+/pCK+ serosal mesothelium present or aberrant WT1 expression in the mesenchyme but not outer layer. 1: partial WT1+/pCK+ serosal mesothelium on the outside of the HIO but not covering the entire organoid. 2: WT1+/pCK+ serosal mesothelium covering the entire HIO. 3: complete or partial WT1+/pCK+ serosal mesothelium on the outside of the HIO combined with ectopic WT1 expression throughout the mesenchyme.

### Single Cell Preparation of Tissue for Single Cell RNA Sequencing

Previously published methods were utilized to carry out cell dissociations (Miller et al., 2020). Tubes and pipette tips were washed with HBSS containing 1% BSA prior to dissociation protocol to prevent adhesion of cell suspensions to the plastic. Centrifugation steps were carried out at 10 °C unless otherwise stated. The Neural Tissue Dissociation Kit (Miltenyi, cat. no. 130-092-628) was used for all RNA-seq dissociations.

Human fetal intestinal tissue or human intestinal organoids was dissociated into single cells by first mechanically cutting tissue into small pieces using a scalpel in a Petri dish containing cold 1×HBSS with Mg²⁺, Ca². Tissue fragments were then transferred to a 15 mL conical tube and treated with Mix 1 for 15 minutes 10 °C. Mix 2 was added to the tube and the remainder of the digestion was carried out at 10 °C until tissue was fully digested. Every 10 minutes, the cell solution was pipetted up and down with a P1000 and assessed under a stereo microscope to determine whether the digestion was complete. Once tissue was digested into a single-cell suspension, cells were passed through a 70 μm filter coated with 1% BSA in 1×HBSS. The filtered cell suspension was centrifuged for 5 minutes at 500 g, and the cell pellet was resuspended in 500 μL 1×HBSS with Mg²⁺, Ca²⁺. 1 mL Red Blood Cell Lysis buffer (Roche cat. No 11814389001) was added to the cell suspension and the tube containing cells was placed on a rocker at 4 °C for 15 minutes. The cell suspension was centrifuged for 5 minutes at 500 g, and the pellet was washed by resuspending in 2 mL HBSS with 1% BSA. Centrifugation and re-suspension were repeated such that cells were washed twice and then counted with a hemocytometer. Cells were then centrifuged and resuspended to reach a final concentration of 1000 cells/μL and kept on ice while transferred to the University of Michigan Advanced Genomics Core where single cell droplets were immediately prepared on the 10× Chromium according to manufacturer instructions. A target of 5,000-10,000 cells captured was utilized. The Chromium Next GEM Single Cell 3′ Library Construction Kit v3.1 was used to prepare single cell libraries according to manufacturer instructions.

RNA Extraction and quantitative RT-PCR Analysis qRT-PCR experiments were carried out as previously described (Miller et al., 2018). RNA was extracted using the MagMAX-96 Total RNA Isolation System (Life Technologies) or Qiagen RNeasy Mini kit for FACS samples. A Nanodrop 2000 spectrophotometer (Thermo Scientific) was used to assess RNA quality and concentration, and then a cDNA library was generated from isolated RNA using the SuperScript VILO cDNA master mix kit (Invitrogen. qRT-PCR analysis was conducted using Quantitect SYBR Green Master Mix (Qiagen) on a Step One Plus Real-Time PCR system (Life Technologies). Gene expression levels were calculated as a change relative to GAPDH or ECAD expression using arbitrary units, which were calculated by the following equation: [2\hat{ }(GAPDH/ECAD Ct-Gene Ct)]×10,000. Expression was normalized to ECAD for epithelial genes as there were variable levels of epithelium between samples. A Ct value of 40 or greater was considered not detectable. A list of primer sequences used can be found in the Table 1.

TABLE 1

| | | |
|---|---|---|
| REAGENT or RESOURCE | SOURCE | IDENTIFIER |

Antibodies

| | | |
|---|---|---|
| Goat polyclonal anti-E-cadherin | R&D Systems | Cat#AF748; RRID: |
| | | AB_355568 |
| Mouse monoclonal anti-E-cadherin | BD Transduction Laboratories | Cat#610181; RRID: |
| | | AB_397580 |
| Goat monoclonal anti-Vimentin | R&D Systems | Cat#MAB2105; |
| | | RRID: AB_2241653 |
| Mouse monoclonal anti-CDX2 | BioGenex | Cat#MU392A-UC; |
| | | RRID: AB_2650531 |
| Rabbit monoclonal anti-PDX1 | Cell Signaling Technology | Cat#5679; RRID: |
| | | AB_10706174 |
| Rabbit monoclonal anti-ZO-1 | Cell Signaling Technology | Cat# 13663; RRID: |
| | | AB_2798287 |
| Rabbit polyclonal anti-MUC2 | Santa Cruz Biotechnology | Cat# sc-15334; |
| | | RRID: AB_2146667 |
| Goat polyclonal anti-CHGA | Santa Cruz Biotechnology | Cat# sc-1488; RRID: |
| | | AB_2276319 |
| Goat polyclonal anti-SOX9 | R&D Systems | Cat# AF3075; RRID: |
| | | AB_2194160 |
| Rabbit monoclonal anti-KI67 | Thermo Scientific | Cat# RM-9106-S1; |
| | | RRID: AB_149792 |
| Goat polyclonal anti-DPPIV | R&D Systems | Cat# AF954; RRID: |
| | | AB_355739 |
| Goat polyclonal anti-LYZ | Santa Cruz Biotechnology | Cat# sc-27958; |
| | | RRID: AB_2138790 |
| Rabbit monoclonal anti-DEFA5 | Abcam | Cat# ab180515 |
| Rabbit polyclonal anti-OLFM4 | Abcam | Cat# ab85046; |
| | | RRID: AB_10670544 |
| Mouse monoclonal anti-αSMA-Cy3 | Sigma-Aldrich | Cat# C6198; RRID: |
| | | AB_476856 |
| Rabbit monoclonal anti-WT1 | Abcam | Cat# ab89901; |
| | | RRID: AB_2043201 |
| Mouse monoclonal anti-pan Cytokeratin | Abcam | Cat# ab86734; |
| | | RRID: AB_10674321 |
| Rabbit polyclonal-anti Laminin | Abcam | Cat# ab11575; |
| | | RRID: AB_298179 |
| Rabbit polyclonal anti-EpCAM | Sigma-Aldrich | Cat# HPA026761; |
| | | RRID: AB_1848198 |
| Rabbit polyclonal anti-PDPN | Santa Cruz Biotechnology | Cat# sc-134482; |
| | | RRID: AB_2162079 |
| Rabbit polyclonal anti-CAV1 | Atlas Antibodies | Cat# HPA049326; |
| | | RRID: AB_2680714 |
| Rabbit polyclonal anti-CAV2 | Atlas Antibodies | Cat# HPA044810; |
| | | RRID: AB_2679098 |
| Mouse monoclonal anti-Ezrin | Sigma-Aldrich | Cat# E8897; RRID: |
| | | AB_476955 |
| Goat polyclonal anti-GATA4 | Sana Cruz Biotechnology | Cat# sc-1237; RRID: |
| | | AB 2108747 |
| Monoclonal anti-ECAD-PE | Miltenyi Biotec | Cat# 130-111-992; |
| | | RRID: AB_2657482 |
| Monoclonal anti-EpCAM-FITC | Miltenyi Biotec | Cat# 130-111-115; |
| | | RRID: AB_2657492 |
| Monoclonal anti-PDPN-APC | Miltenyi Biotec | Cat# 130-107-016; |
| | | RRID: AB_2653263 |
| Donkey anti-mouse 488 | Jackson ImmunoResearch | Cat#715-545-150; |
| | | RRID: AB_2340846 |
| Donkey anti-mouse Cy3 | Jackson ImmunoResearch | Cat#715-165-150; |
| | | RRID: AB_2340813 |
| Donkey anti-mouse 647 | Jackson ImmunoResearch | Cat#715-605-150; |
| | | RRID: AB_2340862 |
| Donkey anti-rabbit 488 | Jackson ImmunoResearch | Cat#711-545-152; |
| | | RRID: AB_2313584 |
| Donkey anti-rabbit Cy3 | Jackson ImmunoResearch | Cat#711-165-152; |
| | | RRID: AB_2307443 |
| Donkey anti-rabbit 647 | Jackson ImmunoResearch | Cat#711-605-152 |
| | | RRID: AB_2492288 |
| Donkey anti-goat 488 | Jackson ImmunoResearch | Cat#705-545-147; |
| | | RRID: AB_2336933 |
| Donkey anti-goat Cy3 | Jackson ImmunoResearch | Cat#705-165-147; |
| | | RRID: AB_2307351 |
| Donkey anti-goat 647 | Jackson ImmunoResearch | Cat#705-605-147; |
| | | RRID: AB_2340437 |

Biological Samples

| | | |
|---|---|---|
| Human Fetal Small Intestine | University of Washington | N/A |
| | Laboratory of Developmental | |
| | Biology | |

Chemicals, Peptides, and Recombinant Proteins

| | | |
|---|---|---|
| Activin A | R&D | Cat#338-AC |
| FGF4 | Purified in house | (Sugawara et al., |
| | | 2014) |
| CHIR99021 | APExBIO | Cat# A3011 |
| Epidermal Growth Factor (EGF) | R&D Systems | Cat# 236-EG |
| Recombinant Human Noggin-FC, purified from | Purified in house | (Heijmans et al., |
| HEK293 cells expressing FC-tagged Noggin | | 2013) |
| Human R-Spondin1 Conditioned Medium from | R&D Systems | Cat# 3710-001-01 |
| Cultrex HA-R-Spondin1-Fc 293 T Cells | | |
| B27 supplement | Thermo Fisher | Cat#17504044 |
| HEPES | Thermo Fisher | Cat#15630080 |
| GlutaMAX | Gibco | Cat#35050061 |
| Penicillin-Streptomycin | Gibco | Cat# 15070063 |
| Dispase | Life Technologies | Cat#17105-041 |
| Poly (2-hydroxyethyl methacrylate) | Sigma-Aldrich | Cat#P3932 |
| TrypLE Express Enzyme | Gibco | Cat# 12604013 |
| Hydrocortisone | STEMCELL Technologies | Cat#07904 |
| Y27632 | Stemgent | Cat#04-0012 |
| SAG | STEMCELL Technologies | Cat#73414 |
| Cyclopamine | Selleckchem | Cat#S1146 |
| IWR1 | R&D Systems | Cat#3532 |
| SU5402 | Santa Cruz Biotechnology | Cat#215543-92-3 |
| DAPT | Calbiochem | Cat#565784 |
| SHH | R&D Systems | Cat#1845-SH |
| WNT3A | R&D Systems | Cat# 5036-WN |
| Collagenase Type II | Life Technologies | Cat#17101-015 |
| Red Blood Cell Lysis Buffer | Roche | Cat#11814389001 |

Critical Commercial Assays

| | | |
|---|---|---|
| Neural Tissue Dissociation Kit (P) | Miltenyi | Cat#130-092-628 |
| RNAscope Multiplex Fluorescent Reagent Kit v2 | ACD | Cat#323100 |
| SuperScript VILO cDNA Synthesis Kit | ThermoFisher | Cat#11754250 |
| MagMAX-96 Total RNA Isolation Kit | Ambion | Cat#AM1830 |
| QuantiTect SYBR Green PCR Kit | Qiagen | Cat#204145 |
| SMARTer Stranded Total RNA-Seq Kit v2- Pico | Takara | Cat#634412 |
| Input | | |
| Chromium Next GEM Single Cell 3′ Library | 10x Genomics | Cat#PN-1000075 |
| Construction Kit v3 | | |
| Chromium Next GEM Single Cell 3′ Library | 10x Genomics | Cat#PN-120237 |
| Construction Kit v2 | | |
| Human t-PA ELISA Kit | ThermoFisher | Cat#BMS258-2 |

Deposited Data

| | | |
|---|---|---|
| Raw scRNA-seq data (human fetal duodenum) | Holloway et al., | ArrayExpress: E- |
| | 2021(Holloway et al., 2021) | MTAB-9489 |
| Raw scRNA-seq data (human fetal small | Holloway et al., 2020 | ArrayExpress: E- |
| intestine) | | MTAB-9363 |
| Raw scRNA-seq data (human fetal small | This study | Deposition of data to |
| intestine) | | ArrayExpress is in |
| | | progress |
| Raw scRNA-seq data (HIO) | Holloway et al., 2020 | ArrayExpress: E- |
| | | MTAB-9228 |
| Raw scRNA-seq data (HIO) | This study | Deposition of data to |
| | | ArrayExpress is in |
| | | progress |

Experimental Models: Cell Lines

| | | |
|---|---|---|
| H9 ESC | WiCell | NIH registry #0062, |
| | | RRID: CVCL_9773 |
| UM 63-1 ESC | MStem Cell Laboratories | NIH registry #0277, |
| | | RRID: CVCL_R782 |
| iPSC 72.3 | Cincinnati Children's Hospital | N/A |
| iPSC WTC11 | Coriell Institute | RRID: CVCL_Y803 |

Oligonucleotides

| | | |
|---|---|---|
| Primer: GAPDH Forward: | IDT | N/A |
| CTCTGCTCCTCCTGTTCGAC | | |
| Primer: GAPDH Reverse: | IDT | N/A |
| TTAAAAGCAGCCCTGGTGAC | | |
| Primer: ECAD Forward: | IDT | N/A |
| TTGACGCCGAGAGCTACAC | | |
| Primer: ECAD Reverse: | IDT | N/A |
| GACCGGTGCAATCTTCAAA | | |
| Primer: CDX2 Forward: | IDT | N/A |
| GGGCTCTCTGAGAGGCAGGT | | |
| Primer: CDX2 Reverse: | IDT | N/A |
| GGTGACGGTGGGGTTTAGCA | | |
| Primer: PDX1 Forward: | IDT | N/A |
| CGTCCGCTTGTTCTCCTC | | |
| Primer: PDX1 Reverse: | IDT | N/A |
| CCTTTCCCATGGATGAAGTC | | |
| Primer: MUC2 Forward: | IDT | N/A |
| TGTAGGCATCGCTCTTCTCA | | |
| Primer: MUC2 Reverse: | IDT | N/A |
| GACACCATCTACCTCACCCG | | |
| | IDT | N/A |

Software and Algorithms

| | | |
|---|---|---|
| ImageJ | Schneider et al., 2012 | https://imagej.nih.gov/ij/ |
| Prism 8.3.0 | GraphPad | https://www.graphpad.com/scientific-software/prism/ |
| Scanpy, Ingest | (Wolf et al., 2018) | https://github.com/theislab/scanpy |
| BBKNN | (Polański et al., 2020) | https://github.com/Teichlab/bbknn |
| Cellranger | 10x Genomics | https://support.10xgenomics.com/single-cell-gene-expression/soft- |
| | | ware/pipelines/latest/what-is-cell-ranger |
| UMAP | (McInnes et al., 2018) | https://github.com/Imcinnes/umap |
| Python 3.7.3 | Python | Python.org |
| Detailed methods and code for scRNAseq | GitHub | https://github.com/jason-spence-lab/Capeling_2022 |
| analysis | | |

Other

| | | |
|---|---|---|
| Matrigel | Corning | Cat#354234 |
| Alginate | Alfa Aesar | Cat# B25266 |
| Histoclear II | National Diagnostics | Cat#HS-202 |

### Tissue Preparation, Immunohistochemistry, and Imaging

### Paraffin Sectioning and Staining

HIO and tHIO tissues were fixed in 4% Paraformaldehyde (Sigma) overnight, washed with PBS, and then dehydrated in an alcohol series: 30 minutes each in 25%, 50%, 75% Methanol: PBS/0.05% Tween-20, followed by 100% Methanol, 100% Ethanol and 70% Ethanol. Tissue was processed into paraffin using an automated tissue processor (Leica ASP300). Paraffin blocks were sectioned 7 μM thick, and immunohistochemical staining was performed as previously described (Spence et al., 2009). Briefly, slides were rehydrated in a series of HistoClear, 100% Ethanol, 95% Ethanol, 70% Ethanol, 30% Ethanol, DI H₂O with 2 changes of 3 minutes each. Antigen retrieval as performed in 1× sodium citrate buffer in a vegetable steamer for 40 minutes. Following antigen retrieval, slides were washed in PBS and permeabilized for 10 minutes in 0.1% TritonX-100 in 1×PBS, blocked for 45 minutes in 0.1% Tween-20, 5% normal donkey serum in 1×PBS. Antibodies used in this study can be found in the Key Resources Table. Primary antibodies were diluted in block and applied overnight at 4 °C. Slides were then washed 3 times in 1×PBS. Secondary antibodies and DAPI were diluted in block and applied for 40 minutes at room temperature. Slides were then washed 3 times in 1×PBS and cover slipped with ProLong Gold.

H&E staining was performed using Harris Modified Hematoxylin (FisherScientific) and Shandon Eosin Y (ThermoScientific) according to manufacturer's instructions. Alcian blue/PAS staining was performed using the Newcomer supply Alcian Blue/PAS Stain kit (Newcomer Supply, Inc.) according to manufacturer's instructions. Trichrome staining was performed by the University of Michigan in vivo Animal Core.

### Imaging and Image Processing

Fluorescently stained slides were imaged on a Nikon A-1 confocal microscope. Brightness and contrast adjustments were carried out using ImageJ (National Institute of Health, USA) (Schneider et al., 2012) and adjustments were made uniformly across images.

### Transmission Electron Microscopy

Suspension HIOs cultured for 28 days were collected for TEM and prepared using conventional TEM sample preparation methods described by the University of Michigan BRCF Microscopy and Image Analysis Laboratory. HIOs were fixed in 3% glutaraldehyde+3% paraformaldehyde in 0.1M cacodylate buffer (CB), pH 7.2 until ready for sample prep. Samples were then washed 3 times for 15 minutes in 0.1M CB. After washing, samples were processed for 1 hour on ice in a post-fixation solution of 1.5% K₄Fe(CN)₆+2% OsO₄ in 0.1M CB. Samples were then washed 3 times in 0.1M CB, and 3 times in 0.1M Na₂+Acetate Buffer, pH 5.2, followed by en bloc staining for 1 hour in 2% Uranyl Acetate+0.1M Na₂+Acetate Buffer, pH 5.2. Samples were then processed overnight in an automated tissue processor, including dehydration from H₂O through 30%, 50%, 70%, 80%, 90%, 95%, 100% ethanol, followed by 100% acetone. Samples were infiltrated with Spurr's resin at a ratio of acetone: Spurr's resin of 2:1 for 1 hour, 1:1 for 2 hours, 1:2 for 16 hours, and absolute Spurr's resin for 24 hours. After embedding and polymerization, samples were sectioned on an ultramicrotome. TEM sample grids were imaged on a JEOL JEM 1400 PLUS TEM.

ELISA for t-PA Expression

HIOs were cultured in Matrigel or suspension for 28 days in vitro. Conditioned media was collected from n>3 wells of HIOs after 28 days and stored at −80 °C until testing. Expression of human t-PA in the conditioned media was detected using a human t-PA ELISA kit (ThermoFisher) according to manufacturer's instructions. The absorbance of each microwell was read on a Molecular Devices SpectraMax M5e microplate reader, using 450 nm as the primary wave length.

### Quantification and Statistical Analysis

Statistical analyses and plots were generated in Prism 8 software (GraphPad). If more than two groups were being compared within a single experiment, an unpaired one-way ANOVA was performed followed by Tukey's multiple comparisons test to compare the mean of each group with the mean of every other group within the experiment unless otherwise specified. For all statistical tests, a significance value of 0.05 was used. For every analysis, the strength of p values is reported in the figures according the following: p>0.05, *p≤0.05, **p≤0.01, ***p≤0.001, ****p≤0.0001. Details of statistical tests can be found in the figure legends. With the exception of scRNA-seq, three hPSC lines were used across experiments with at least 3 independent experiments and at least 3 technical replicates per experiment.

### Computational Analysis of Single-Cell RNA Sequencing Data

### Overview

To visualize distinct cell populations within the single-cell RNA sequencing dataset, we employed the general workflow outlined by the Scanpy Python package (Wolf et al., 2018). This pipeline includes the following steps: filtering cells for quality control, log normalization of counts per cell, extraction of highly variable genes, regressing out specified variables, scaling, reducing dimensionality with principal component analysis (PCA) and uniform manifold approximation and projection (UMAP) (Becht et al., 2019; McInnes et al., 2018), and clustering by the Louvain algorithm (Blondel et al., 2008).

Sequencing Data and Processing FASTQ Reads into Gene Expression Matrices

All single-cell RNA sequencing was performed at the University of Michigan Advanced Genomics Core with an Illumina Novaseq 6000. The 10× Genomics Cell Ranger pipeline was used to process raw Illumina base calls (BCLs) into gene expression matrices. BCL files were demultiplexed to trim adaptor sequences and unique molecular identifiers (UMIs) from reads. Each sample was then aligned to the human reference genome (hg19) to create a filtered feature bar code matrix that contains only the detectable genes for each sample.

### Quality Control

To ensure quality of the data, all samples were filtered to remove cells expressing too few or too many genes (FIGS. 3C-E/FIG. 4A—<500, >10000; FIGS. 4B-C/FIG. 3F—<500, >8000; FIG. 6/FIG. 5—<500, >8000; FIG. 5F,G/FIG. 8A—<500, >10000), with high UMI counts (FIG. 3C-E/FIG. 4A—60000; FIG. 4B-C/FIG. 3F—50000; FIG. 6/FIG. 5—50000; FIG. 5F,G/FIG. 8A—60000), or a fraction of mitochondrial genes greater than 0.1.

### Normalization and Scaling

Data matrix read counts per cell were log normalized, and highly variable genes were extracted. Using Scanpy's simple linear regression functionality, the effects of total reads per cell and mitochondrial transcript fraction were removed. The output was then scaled by a z-transformation. Following these steps, a total of (FIG. 3C-E/FIG. 4A—51790 cells, 1710 genes; FIG. 4B-C/FIG. 3F—4619 cells, 4167 genes; FIG. 6/FIG. 5—13055 cells, 2117 genes; FIG. 5F,G/FIG. 8A—52689 cells, 2615 genes) were kept for clustering and visualization.

### Variable Gene Selection

Highly variable genes were selected by splitting genes into 20 equal-width bins based on log normalized mean expression. Normalized variance-to-mean dispersion values were calculated for each bin. Genes with log normalized mean expression levels between 0.125 and 3 and normalized dispersion values above 0.5 were considered highly variable and extracted for downstream analysis.

### Batch Correction

We have noticed batch effects when clustering data due to technical artifacts such as timing of data acquisition or differences in dissociation protocol. To mitigate these effects, we used the Python package BBKNN (batch balanced k nearest neighbors) (Polański et al., 2020). BBKNN was selected over other batch correction algorithms due to its compatibility with Scanpy and optimal scaling with large datasets. This tool was used in place of Scanpy's nearest neighbor embedding functionality. BBKNN uses a modified procedure to the k nearest neighbors' algorithm by first splitting the dataset into batches defined by technical artifacts. For each cell, the nearest neighbors are then computed independently per batch rather than finding the nearest neighbors for each cell in the entire dataset. This helps to form connections between similar cells in different batches without altering the PCA space. After completion of batch correction, cell clustering should no longer be driven by technical artifacts.

### Dimension Reduction and Clustering

Principal component analysis (PCA) was conducted on the filtered expression matrix followed. Using the top principal components, a neighborhood graph was calculated for the nearest neighbors (FIG. 3C-E/FIG. 4A—16 principal components, 30 neighbors; FIG. 4B-C/FIG. 3F—12 principal components, 15 neighbors; FIG. 6/FIG. 5—12 principal components, 15 neighbors; FIG. 5F,G/FIG. 8A—16 principal components, 30 neighbors). BBKNN was implemented when necessary and calculated using the top 50 principal components with 3 neighbors per batch. The UMAP algorithm was then applied for visualization on 2 dimensions. Using the Louvain algorithm, clusters were identified with a resolution of (FIG. 3C-E/FIG. 4A—0.8; FIG. 4B-C/FIG. 3F—0.35; FIG. 6/FIG. 5—0.65, FIG. 5F,G/FIG. 8A—1).

### Cluster Annotation

Using canonically expressed gene markers, each cluster's general cell identity was annotated. Markers utilized include epithelium (CDH1, EPCAM, CDX2, PDX1, VIL1, CLDN4), mesenchyme (VIM, COL1A2, PDGFRA, DCN, TCF21, COL3A1, FOXF1), neuronal (POSTN, S100B, STMN2, ELAV4), endothelial (ESAM, CDH5, CD34, KDR), immune (CD53, VAMP8, CD48, ITGB2), and serosal mesothelium (MSLN, WT1, UPK3B, PDPN).

### Sub-Clustering

After annotating clusters within the UMAP embedding, specific clusters of interest were identified for further sub-clustering and analysis. The corresponding cells were extracted from the original filtered but unnormalized data matrix to include (FIG. 4A—1040 cells, FIG. 4C—962 cells, FIG. 6B/C—1903 cells, FIG. 5G, FIG. 8A—8179). The extracted cell matrix then underwent log normalization, variable gene extraction, linear regression, z transformation, and dimension reduction to obtain a 2-dimensional UMAP embedding for visualization.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes.

Complete citations for the references cited within the application are provided within the following reference list. Indeed, each of the following references are herein incorporated by reference in their entireties:
Arora, N., Alsous, J. I., Guggenheim, J. W., Mak, M., Munera, J., Wells, J. M., Kamm, R. D., Asada, H. H., Shvartsman, S. Y., and Griffith, L. G. (2017). A process engineering approach to increase organoid yield. Development 144, 1128-1136.
Becht, E., McInnes, L., Healy, J., Dutertre, C.-A., Kwok, I. W. H., Ng, L. G., Ginhoux, F., and Newell, E. W. (2019). Dimensionality reduction for visualizing single-cell data using UMAP. Nature Biotechnology 37, 38-44.
Blondel, V. D., Guillaume, J.-L., Lambiotte, R., and Lefebvre, E. (2008). Fast unfolding of communities in large networks. J Stat Mech.
Bloom, W., and Fawcett, D. W. (1975). A textbook of histology, 10th edition (Phildadelphia, PA, USA: Saunders), pp. 1033.
Bridda, A., Padoan, I., Mencarelli, R., and Frego, M. (2007). Peritoneal Mesothelioma: A Review. Medscape General Medicine 9, 32.
Capeling, M., Czerwinski, M., Huang, S., Tsai, Y.-H., Wu, A., Nagy, M. S., Juliar, B., Sundaram, N., Song, Y., Han, W. M., et al. (2019). Nonadhesive Alginate Hydrogels Support Growth of Pluripotent Stem Cell-Derived Intestinal Organoids. Stem Cell Reports 12, 381-394.
Capeling, M., Huang, S., Mulero-Russe, A., Cieza, R., Tsai, Y.-H., Garcia, A. J., and Hill, D. R. (2020). Generation of small intestinal organoids for experimental intestinal physiology. Methods Cell Biol 159, 143-174.
Carmona, R., Cano, E., Mattiotti, A., Gaztambide, J., and Muñoz-Chápuli, R. (2013). Cells Derived from the Coelomic Epithelium Contribute to Multiple Gastrointestinal Tissues in Mouse Embryos. PLOS One 8, e55890.
Chan, R., Edwards, B., Hu, R., Rossitto, P., Min, B., Lund, J., and Cardiff, R. (1988). Characterization of two monoclonal antibodies in an immunohistochemical study of keratin 8 and 18 expression. Am J Clin Pathol 89, 472-480.
Choi, K., Vodyanik, M., and Slukvin, I. (2011). Hematopoietic differentiation and production of mature myeloid cells from human pluripotent stem cells. Nature Protocols 6, 296-313.
Colunga, T., Hayworth, M., Kreß, S., Reynolds, D. M., Chen, L., Nazor, K. L., Baur, J., Singh, A. M., Loring, J. F., Metzger, M., et al. (2019). Human Pluripotent Stem Cell-Derived Multipotent Vascular Progenitors of the Mesothelium Lineage Have Utility in Tissue Engineering and Repair. Cell Reports 26, 2566-2579.
Czerwinski, M., and Spence, J. R. (2017). Hacking the Matrix. Cell Stem Cell 20, 9-10.
Dedhia, P. H., Bertaux-Skeirik, N., Zavros, Y., and Spence, J. R. (2016). Organoid Models of Human Gastrointestinal Development and Disease. Gastroenterology 150, 1098-1112.
Dettman, R. W., Denetclaw Jr., W., Ordahl, C. P., and Bristow, J. (1998). Common epicardial origin of coronary vascular smooth muscle, perivascular fibroblasts, and intermyocardial fibroblasts in the avian heart. Developmental Biology 193, 169-181.
Dixit, R., Ai, X., and Fine, A. (2013). Derivation of lung mesenchymal lineages from the fetal mesothelium requires hedgehog signaling for mesothelial cell entry Development 140, 4398-4406.
Dobbie, J. (1992). Pathogenesis of peritoneal fibrosing syndromes (sclerosing peritonitis) in peritoneal dialysis. Peritoneal Dialysis International 12, 14-27.
Federici, S., Domenichelli, V., Antonelli, C., and Domini, R. (2003). Multiple intestinal atresia with apple peel syndrome: successful treatment by five end-to-end anastomoses, jejunostomy, and transanastomotic silicone stent. Journal of Pediatric Surgery 38, 1250-1252.
Finkbeiner, S. R., Hill, D. R., Altheim, C. H., Dedhia, P. H., Taylor, M. J., Tsai, Y.-H., Chin, A. M., Mahe, M. M., Watson, C. L., Freeman, J. J., et al. (2015). Transcriptome-wide Analysis Reveals Hallmarks of Human Intestine Development and Maturation In Vitro and In Vivo. Stem Cell Reports 4, 1140-1155.
Foley-Comer, A. J., Herrick, S. E., Al-Mishlab, T., M. Prêle, C., Laurent, G. J., and Mutsaers, S. E. (2002). Evidence for incorporation of free-floating mesothelial cells as a mechanism of serosal healing. Journal of Cell Science 115, 1383-1389.
Heijmans, J., van Lidth de Jeude, J., Koo, B., Rosekrans, S., Wielenga, M., van de Wetering, M., Ferrante, M., Lee, A., Onderwater, J., Paton, J., et al. (2013). ER stress causes rapid loss of intestinal epithelial stemness through activation of the unfolded protein response. Cell Reports 3, 1128-1139.
Herrick, S. E., and Mutsaers, S. E. (2007). The Potential of Mesothelial Cells in Tissue Engineering and Regenerative Medicine Applications. Int J Artif Organs 30, 527-540.
Hill, D. R., Huang, S., Nagy, M. S., Yadagiri, V. K., Fields, C., Muckherjee, D., Bons, B., Dedhia, P. H., Chin, A. M., Tsai, Y.-H., et al. (2017). Bacterial colonization stimulates a complex physiological response in the immature human intestinal epithelium. eLife 6.
Hohwieler, M., Illing, A., Hermann, P. C., Mayer, T., Stockmann, M., Perkhofer, L., Eiseler, T., Antony, J. S., Müller, M., Renz, S., et al. (2017). Human pluripotent stem cell-derived acinar/ductal organoids generate human pancreas upon orthotopic transplantation and allow disease modelling. Gut 66, 473-486.
Holloway, E. M., Czerwinski, M., Tsai, Y.-H., Wu, J., Wu, A., Childs, C., Walton, K. D., Sweet, C., Yu, Q., Glass, I., et al. (2021). Mapping Development of the Human Intestinal Niche at Single-Cell Resolution. Cell Stem Cell 28, 568-580.e564.
Holloway, E. M., Wu, J. H., Czerwinski, M., Sweet, C. W., Wu, A., Tsai, Y.-H., Huang, S., Stoddard, A. E., Capeling, M. M., Glass, I., et al. (2020). Differentiation of Human Intestinal Organoids with Endogenous Vascular Endothelial Cells. Developmental Cell 54, 516-528.e517.
Inagaki, N. F., Inagaki, F. F., Kokudo, N., and Miyajima, A. (2019). Generation of mesothelial progenitor-like cells from mouse-induced pluripotent stem cells. FEBS Letters 593, 386-394.
Josephson, R., Sykes, G., Liu, Y., Ording, C., Xu, W., Zeng, X., Shin, S., Loring, J., Maitra, A., Rao, M. S., et al. (2006). A molecular scheme for improved characterization of human embryonic stem cell lines. BMC Biology 4.
Kanamori-Katayama, M., Kaiho, A., Ishizu, Y., Okamura-Oho, Y., Hino, O., Abe, M., Kishimoto, T., Sekihara, H., Nakamura, Y., Suzuki, H., et al. (2011). LRRN4 and UPK3B are markers of primary mesothelial cells. PLOS One 6.
Kawaguchi, M., Bader, D. M., and Wilm, B. (2007). Sesrosal Mesothelium Retains Vasculogenic Potential. Developmental Dynamics 236, 2973-2979.
Kolterud, A., Grosse, A. S., Zacharias, W. J., Walton, K. D., Kretovich, K. E., Madison, B. B., Waghray, M., Ferris, J. E., Hu, C., Merchant, J. L., et al. (2009). Paracrine Hedgehog signaling in stomach and intestine: new roles for hedgehog in gastrointestinal patterning. Gastroenterology 137, 618-628.
Kruithof, B. P. T., van Wijk, B., Somi, S., Kruithof-de Julio, M., Pomares, J. M. P., Weesie, F., Wessels, A., Moorman, A. F. M., and van den Hoff, M. J. B. (2006). BMP and FGF regulate the differentiation of multipotential pericardial mesoderm into the myocardial or epicardial lineage. Developmental Biology 295, 507-522.
Kumar-Singh, S., Segers, K., Rodeck, U., Backhovens, H., Bogers, J., Weyler, J., Van Broeckhoven, C., and Van Marck, E. (1997). WT1 mutation in malignant mesothelioma and WT1 immunoreactivity in relation to p53 and growth factor receptor expression, cell-type transition, and prognosis. J Pathol 181, 67-74.
Kumar, S. V., Er, P. X., Lawlor, K. T., Motazedian, A., Scurr, M., Ghobrial, I., Combes, A. N., Zappia, L., Oshlack, A., Stanley, E. G., et al. (2019). Kidney micro-organoids in suspension culture as a scalable source of human pluripotent stem cell-derived kidney cells. Development 146, dev172361.
Lachaud, C. C., Rodriguez-Campins, B., Hmadcha, A., and Soria, B. (2015). Use of mesothelial cells and biological matrices for tissue engineering of simple epithelium surrogates. Frontiers in Bioengineering and Biotechnology 3.
Lua, I., Li, Y., Pappoe, L. S., and Asahina, K. (2015). Myofibroblastic Conversion and Regeneration of Mesothelial Cells in Peritoneal and Liver Fibrosis. Am J Pathool 185, 3258-3273.
Matsuo, Y., Nishizaki, C., and Drexler, H. (1999). Efficient DNA fingerprinting method for the identification of cross-culture contamination of cell lines. Human Cell 12, 149-154.
McCracken, K., Howell, J. C., Wells, J. M., and Spence, J. R. (2011). Generating human intestinal tissue from pluripotent stem cells in vitro. Nature Protocols 6, 1920-1928.
McCracken, K. W., Catá, E. M., Crawford, C. M., Sinagoga, K. L., Schumacher, M., Rockich, B. E., Tsai, Y.-H., Mayhew, C. N., Spence, J. R., Zavros, Y., et al. (2014). Modelling human development and disease in pluripotent stem-cell-derived gastric organoids. Nature 516, 400-404.
McInnes, L., Healy, J., and Melville, J. (2018). UMAP: Uniform Manifold Approximation and Projection for Dimension Reduction. arXiv e-prints 1802.03426.
Miller, A. J., Yu, Q., Czerwinski, M., Tsai, Y.-H., Conway, R. F., Wu, A., Holloway, E. M., Walker, T., Glass, I. A., Treutlein, B., et al. (2020). In Vitro and In Vivo Development of the Human Airway at Single-Cell Resolution. Developmental Cell 53, 117-128.e116.
Mutsaers, S. E. (2002). Mesothelial cells: Their structure, function and role in serosal repair. Respirology 7, 171-191.
Mutsaers, S. E. (2004). The mesothelial cell. The International Journal of Biochemistry & Cell Biology 36, 9-16.
Ootani, A., Li, X., Sangiorgi, E., Ho, Q., Ueno, H., Toda, S., Sugihara, H., Fujimoto, K., Weissman, I., Capecchi, M., et al. (2009). Sustained in vitro intestinal epithelial culture within a Wnt-dependent stem cell niche. Nature Medicine 15, 701-706.
Pansky, B. (1982). Review of Medical Embryology.
Polański, K., Young, M. D., Miao, Z., Meyer, K. B., Teichmann, S. A., and Park, J.-E. (2020).
BBKNN: fast batch alignment of single cell transcriptomes. Bioinformatics 36, 964-965.
Pumberger, W., Birnbacher, R., Pomberger, G., and Deutinger, J. (2002). Duodeno-jejunal atresia with volvulus, absent dorsal mesentery, and absent superior mesenteric artery: a hereditary compound structure in duodenal atresia? American journal of Medical Genetics 109, 52-55.
Que, J., Wilm, B., Hasegawa, H., Wang, F., Bader, D. M., and Hogan, B. L. M. (2008). Mesothelium contributes to vascular smooth muscle and mesenchyme during lung development. PNAS 105, 16626-16630.
Rinkevich, Y., Mori, T., Sahoo, D., Xu, P., Bermingham, J. J., and Weissman, I. (2012). Identification and prospective isolation of a mesothelial precursor lineage giving rise to smooth muscle cells and fibroblasts for mammalian internal organs, and their vasculature. Nature Cell Biology 14, 1251-1260.
Rudat, C., Grieskamp, T., Röhr, C., Airik, R., Wrede, C., Hegermann, J., Herrmann, B. G., Schuster-Gossler, K., and Kispert, A. (2014). Upk3b Is Dispensable for Development and Integrity of Urothelium and Mesothelium. PLOS One 9.
Schlieve, C. R., Fowler, K. L., Thornton, M., Huang, S., Hajjali, I., Hou, X., Grubbs, B., Spence, J. R., and Grikscheit, T. C. (2017). Neural Crest Cell Implantation Restores Enteric Nervous System Function and Alters the Gastrointestinal Transcriptome in Human Tissue-Engineered Small Intestine. Stem Cell Reports 9, 883-896.
Schlueter, J., Manner, J., and Brand, T. (2006). BMP is an important regulator of proepicardial identity in the chick embryo. Developmental Biology 295, 546-558.
Schneider, C. A., Rasband, W. S., and Eliceiri, K. W. (2012). NIH Image to ImageJ: 25 years of image analysis. Nature Methods 9, 671-675
Spence, J. R., Lange, A. W., Lin, S.-C. J., Kaestner, K. H., Lowy, A. M., Kim, I., Whitsett, J. A., and Wells, J. M. (2009). Sox17 regulates organ lineage segregation of ventral foregut progenitor cells. Developmental Cell 17, 62-74.
Spence, J. R., Mayhew, C. N., Rankin, S. A., Kuhar, M., Vallance, J. E., Tolle, K., Hoskins, E. E., Kalinichenko, V. V., Wells, S. I., Zorn, A. M., et al. (2011). Directed differentiation of human pluripotent stem cells into intestinal tissue in vitro. Nature 470, 105-109.
Sugawara, S., Ito, T., Sato, S., Sato, Y., Kojima, I., and Kobayashi, M. (2014). Production of an aminoterminally truncated, stable type of bioactive mouse fibroblast growth factor 4 in Escherichia coli. Journal of Bioscience and Bioengineering 117, 525-530.
Sulaiman, H., Gabell, G., Davis, M. C., Mutsaers, S. E., Boulos, P., Laurent, G. J., and Herrick, S. E. (2001). Presence and distribution of sensory nerve fibers in human peritoneal adhesions. Annals of Surgery 234, 256-261.
Takahashi, Y., Sato, S., Kurashima, Y., Yamamoto, T., Kurokawa, S., Yuki, Y., Takemura, N., Uematsu, S., Lai, C.-Y., Otsu, M., et al. (2018). A Refined Culture System for Human Induced Pluripotent Stem Cell-Derived Intestinal Epithelial Organoids. Stem Cell Reports 10, 314-328.
Thomason, R. T., Bader, D. M., and Winters, N. I. (2012). A Comprehensive Timeline of Mesodermal Development in the Quail Small Intestine. Developmental Dynamics 241, 1678-1694.
Tian, H., Biehs, B., Chiu, C., Siebel, C. W., Wu, Y., Costa, M., de Sauvage, F. J., and Klein, O. D. (2015). Opposing activities of Notch and Wnt signaling regulate intestinal stem cells and gut homeostasis. Cell Reports 11, 33-42.
Tsai, Y.-H., Nattiv, R., Dedhia, P. H., Nagy, M. S., Chin, A. M., Thomson, M., Klein, O. D., and Spence, J. R. (2017). In vitro patterning of pluripotent stem cell-derived intestine recapitulates in vivo human development. Development 144, 1045-1055.
VanDussen, K., Carulli, A., Keeley, T., Patel, S., Puthoff, B., Magness, S., Tran, I., Mailard, I., Siebel, C., Kolterud, A., et al. (2012). Notch signaling modulates proliferation and differentiation of intestinal crypt base columnar stem cells. Development 139.
Verzi, M. P., and Shivdasani, R. A. (2008). Wnt signaling in gut organogenesis. Organogenesis 4, 87-91.
Wada, A. M., Smith, T. K., Osler, M. E., Reese, D. E., and Bader, D. M. (2003). Epicardial/Mesothelial Cell Line Retains Vasculogenic Potential of Embryonic Epicardium. Circulation Research 92, 525-531.
Waldhausen, J. H., and Sawin, R. S. (1997). Improved long-term outcome for patients with jejunoileal apple peel atresia. Journal of Pediatric Surgery 32, 1307-1309.
Watson, C., Mahe, M. M., Munera, J., Howell, J. C., Sundaram, N., Poling, H. M., Schweitzer, J., Vallance, J. E., Mayhew, C. N., Sun, Y., et al. (2014). An in vivo model of human small intestine using pluripotent stem cells. Nature Medicine 20, 1310-1314.
Watt, A. J., Battle, M. A., Li, J., and Duncan, S. A. (2004). GATA4 is essential for formation of the proepicardium and regulates cardiogenesis. PNAS 101, 12573-12578.
Wilm, B., Ipenberg, A., Hastie, N. D., Burch, J. B. E., and Bader, D. M. (2005). The serosal mesothelium is a major source of smooth muscle cells of the gut vasculature. Development 132, 5317-5328.
Winters, N. I., and Bader, D. M. (2013). Development of the Serosal Mesothelium. Journal of Developmental Biology 1, 64-81.
Winters, N. I., Thomason, R. T., and Bader, D. M. (2012). Identification of a novel developmental mechanism in the generation of mesothelia. Development 139, 2926-2934.
Wolf, F. A., Angerer, P., and Theis, F. J. (2018). SCANPY: large-scale single-cell gene expression data analysis. Genome Biology 19.
Workman, M. J., Mahe, M. M., Trisno, S., Poling, H. M., Watson, C. L., Sundaram, N., Chang, C.-F., Schiesser, J., Aubert, P., Stanley, E. G., et al. (2016). Engineered human pluripotent-stem-cell-derived intestinal tissues with a functional enteric nervous system. Nature Medicine 23, 49-59.
Yu, Q., Kilik, U., Holloway, E. M., Tsai, Y.-H., Harmel, C., Wu, A., Wu, J. H., Czerwinski, M., Childs, C., He, Z., et al. (2021). Charting human development using a multi-endodermal organ atlas and organoid models. Cell 184, 3281-3298.e3222.
Yung, S., Li, F. K., and Chan, T. M. (2006). Peritoneal Mesothelial Cell Culture and Biology. Peritoneal Dialysis International 26, 162-173.
Ziprin, P., Ridgway, P. F., Peck, D. H., and Darzi, A. W. (2003). Laparoscopic-type environment enhances mesothelial cell fibrinolytic activity in vitro via a down-regulation of plasminogen activator inhibitor-1 activity. Surgery 134, 758-765.
Heijmans, J., van Lidth de Jeude, J., Koo, B., Rosekrans, S., Wielenga, M., van de Wetering, M., Ferrante, M., Lee, A., Onderwater, J., Paton, J., et al. (2013). ER stress causes rapid loss of intestinal epithelial stemness through activation of the unfolded protein response. Cell Rep 3, 1128-1139.
Holloway, E. M., Czerwinski, M., Tsai, Y.-H., Wu, J., Wu, A., Childs, C., Walton, K. D., Sweet, C., Yu, Q., Glass, I., et al. (2021). Mapping Development of the Human Intestinal Niche at Single-Cell Resolution. Cell Stem Cell 28, 568-580.e564.
McInnes, L., Healy, J., and Melville, J. (2018). UMAP: Uniform Manifold Approximation and Projection for Dimension Reduction. arXiv e-prints 1802.03426.
Sugawara, S., Ito, T., Sato, S., Sato, Y., Kojima, I., and Kobayashi, M. (2014). Production of an aminoterminally truncated, stable type of bioactive mouse fibroblast growth factor 4 in Escherichia coli. Journal of Bioscience and Bioengineering 117, 525-530.
Wolf, F. A., Angerer, P., and Theis, F. J. (2018). SCANPY: large-scale single-cell gene expression data analysis. Genome Biology 19.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method, comprising culturing obtained human intestinal organoids (HIOs) in suspension, wherein the culturing of the obtained HIOs in suspension results in suspension HIOs and in vitro differentiation of an organized serosal mesothelial layer (e.g., suspension HIOs having a serosal mesothelial layer).

2. The method of claim 1, wherein the culturing of the obtained HIOs in suspension comprises seeding the obtained HIOs with a growth medium.

3. The method of claim 2, wherein the growth medium comprises one or more of the following: Advanced Dulbecco's Modified Eagle Medium/Nutrient Mixture F-12 (DMEM/F12) supplemented with 4-(2-hydroxyethyl)-1-piperazineethanesulfonic acid (HEPES), GlutaMAX, Penicillin/Streptomycin, epidermal growth factor (EGF), a BMP inhibiting agent (e.g., Noggin), and R-Spondin.

4. The method of claim 2, wherein the growth medium comprises Advanced DMEM/F12 supplemented with HEPES, GlutaMAX, Penicillin/Streptomycin, EGF, Noggin, and R-Spondin.

5. The method of claim 2, wherein seeding the obtained HIOs with the growth medium comprises plating the obtained HIOs in low attachment plates with the growth medium such that the HIOs remain in suspension instead of adhering to the culture dish.

6. The method of claim 2, wherein seeding the obtained HIOs with the growth medium comprises plating the obtained HIOs in low attachment plates with the growth medium having EGF, Noggin, and R-spondin for approximately 72 hours (e.g., 65 hours, 66, 67, 68, 69, 70, 71, 72, 73, 74, 75, 76, 77, 78, 79 hours) followed by culturing with EGF for remaining culture time.

7. The method of claim 1, wherein the suspension HIOs having a serosal mesothelial layer comprise an inner ECAD+ epithelium surrounded by VIM+ mesenchyme.

8. The method of claim 1, wherein the suspension HIOs having a serosal mesothelial layer comprise expression of one or more of DPP4+ enterocytes, MUC2+ goblet cells, and CHGA+ enteroendocrine cells.

9. The method of claim 1, wherein the suspension HIOs having a serosal mesothelial layer comprise expression of one or more of WT1, UPK3B, MSLN, and KRT19.

10. The method of claim 1, wherein the serosal mesothelial layer of the suspension HIOs express one or more of CAV1, CAV2, and EZR.

11. The method of claim 1, wherein the suspension HIOs having a serosal mesothelial layer comprise an epithelium and/or mesenchyme.

12. The method of claim 11, wherein the epithelium expresses one or both of CDX2 and PDX1.

13. The method of claim 11, wherein the epithelium and mesenchyme express KI67, and/or wherein the epithelium expresses SOX9.

14. The method of claim 1, wherein the suspension HIOs having a serosal mesothelial layer comprise an apical surface.

15. The method of claim 14, wherein the apical surface expresses ZO-1.

16. The method of claim 1, wherein the obtained HIOs are derived from intestinal hindgut spheroids.

17. A composition comprising or consisting suspension HIOs having a serosal mesothelial layer produced in vitro from the method of claim 1.

18. A kit comprising or consisting of suspension HIOs having a serosal mesothelial layer produced in vitro from the method of claim 1.

19. A method of treating a mammalian subject having a damaged intestinal tissue with reduced function by engrafting at the site of injury suspension HIOs having an organized serosal mesothelial layer produced in vitro from the method of claim 1.
